SEQUENCE LISTING (1 sequence)

SEQ ID NO: 1 - DNA, 112 bp, synthetic construct
  misc_feature 1..13: note = DNA
  source 1..112: mol_type = other DNA
  misc_feature 14: note = RNA
  misc_feature 15..112: note = DNA
  misc_binding 1: bound_moiety = Cy5
  misc_binding 112: bound_moiety = AmMO
ctatgaactgactatgacctcactaccaagcaagcatggacaataccgagcctttcatttcagccgatcatacctcaatgtagataagcacatcttgtcatcggaggcttag

(19) United States
(12) Patent Application Publication
(10) Pub. No.: US 2025/0224396 A1
(43) Pub. Date: Jul. 10, 2025

(54) BIOMOLECULE-BASED WRINKLED NETWORK AND RELATED METHODS

(71) Applicant: McMaster University, Hamilton (CA)

(72) Inventors: Zeinab Hosseinidoust, Dundas (CA); Tohid Didar, Dundas (CA); Lei Tian, Hamilton (CA)

(21) Appl. No.: 18/893,282

(22) Filed: Sep. 23, 2024

Related U.S. Application Data
(60) Provisional application No. 63/539,838, filed on Sep. 22, 2023.

(51) Int. Cl. G01N 33/569; C12N 15/11

(52) U.S. Cl. CPC G01N 33/56911; C12N 15/11; C12N 2310/127; G01N 2333/195

(57) ABSTRACT

This disclosure relates to a method for preparing wrinkled structures and uses thereof.

### STATEMENT REGARDING ELECTRONIC FILING OF A SEQUENCE LISTING

A Sequence Listing XML file, submitted under 37 C.F.R. §§ 1.831-835, entitled “16112-37 Seq Listing.xml”, 3,868 bytes in size, and created on Mar. 28, 2025. The Sequence Listing is incorporated herein by reference in its entirety into the specification for its disclosures.

### FIELD

The present disclosure relates to wrinkled networks. More specifically, the present disclosure relates to biomolecule-based wrinkled networks as well as methods of making and using same.

### BACKGROUND

Inspired by nature, hierarchically-structured biomimetic materials have been revolutionizing fields ranging from tissue engineering¹⁻³ to biosensing^{4,5} to (bio)fouling mitigation^{6,7}. Wrinkled materials are among of the most versatile hierarchical structures and have far-reaching applications in wearables⁸, flexible electronics⁹, energy conversion¹⁰, and adhesive/repellent surfaces^{11,12}. In nature, wrinkled structures are made with proteins as the foundational components that drive the structural hierarchy and add nanoarchitecture to the material. Likewise, these foundational components are responsible for the unique functionalities of wrinkled structures that would otherwise be limited without them. Such added capabilities include rapid environmental trigger-response, as is the case of white blood cells¹³, or contractility, as is the case of esophageal mucosa¹⁴. Nature has so far remained the exclusive engineer of such sophisticated structures with biomimicry efforts hindered by the lack of compatible technologies and reliable methodologies that can preserve the nanoarchitecture of proteins and proteinaceous bionanoparticle building blocks. One of the most common methods of inducing wrinkles is utilizing the mismatching deformation in a bilayer system composed of a shrinkable substrate and the attached material. These properties are commonly triggered by mechanical stretching¹⁵, heat^{12,16}, and/or solvents^{17,18}. The heat-induced substrate-shrinkage and solvent-induced swelling-shrinkage can efficiently create wrinkle patterns. In doing so, however, these methods are known to denature proteins and proteinaceous bionanoparticles, thereby preventing the desired nanoarchitecture¹⁹. The substrate stretching/release method is only applicable to very specific substrate-coating combinations, and is commonly accompanied by solvent or heat that cannot be readily integrated into refined microprinting systems¹⁵.

Viral nanoparticles such as bacteriophages (bacterial viruses) are attractive candidates for introducing the missing nanoarchitecture within engineered wrinkled structures. This is largely due to their properties that are unmatched by engineered nanoparticles, namely monodisperse self-replication, remarkable diversity in shape and size, and precise control of surface chemistry through chemical and/or genetic modification^{20,21}. When leveraged together, these properties make bacteriophages powerful natural building blocks for the next generation of biomimetic designs^{22,23} Previous work has demonstrated that nanofilamentous phages are capable of self-organizing at high concentrations, forming 2-dimensional (2D) films²⁴, bulk hydrogels²⁵⁻²⁷, and spheres^{28,29} leading to micro and macro scale materials with powerful optical and structural properties. In total, viral-based material platforms represent the next frontier for creating functional 3D structural materials. Despite the enormous promise, a universal method to induce wrinkled structures in various biomolecule-based materials, including virus-built hierarchical scaffolds, while preserving their nanoarchitecture, remains elusive.

The background herein is included solely to explain the context of the disclosure. This is not to be taken as an admission that any of the material referred to was published, known, or part of the common general knowledge as of the priority date.

### SUMMARY

In accordance with an aspect, there is provided a biomolecule-based wrinkled network.

In an aspect, the wrinkled network is nano-reticular.

In an aspect, the wrinkled network comprises a hierarchical architecture.

In an aspect, the wrinkled network comprises four levels of hierarchy.

In an aspect, the wrinkled network comprises a tunable pattern of wrinkle shape and/or density.

In an aspect, the biomolecule comprises a protein, a nucleic acid, a bacteria, a virus, a phage, or any combination thereof.

In an aspect, the wrinkled network further comprises a cargo.

In an aspect, the cargo is homogenously distributed throughout the wrinkled network.

In an aspect, the cargo comprises a biorecognition element.

In an aspect, the biorecognition element comprises an antibody, an enzyme, an aptamer, a cell, a nucleic acid, a protein, or any combination thereof.

In an aspect, the enzyme comprises a deoxyribozyme.

In an aspect, the biorecognition element detects an enzyme, antibody, antigen, nucleic acid, cell, aptamer, tissue, microorganism, organelle, cell receptor, or any combination thereof.

In an aspect, the wrinkled network is configured in an antimicrobial material, a microarray, a wearable patch, a smart textile, a wearable device, a flexible electronic, an adhesive/repellent surface, a biosensor, a bioassays, a cell culture substrate, a biomimetic material, a surface-enhanced Raman spectroscopy, an electrode, a catalyst, a drug delivery device, or an energy storage material.

In an aspect, there is provided a method for wrinkling a biomolecule-based network disposed on a substrate, the method comprising shrinking the substrate without using heat or solvent.

In an aspect, shrinking the substrate comprises applying high pressure plasticizer gas to the substrate.

In an aspect, the high pressure plasticizer gas comprises carbon dioxide and/or hydrogen.

In an aspect, the biomolecule comprises a protein, a nucleic acid, a bacteria, a virus, a phage, or any combination thereof.

In an aspect, the method further comprises printing the biomolecule-based network on the substrate.

In an aspect, the method further comprises crosslinking the biomolecule-based network prior to shrinking the substrate.

In an aspect, the method further comprises drying the biomolecule-based network prior to shrinking the substrate.

In an aspect, the wrinkle shape, density, and/or homogeneity are tunable by adjusting the hydrophobicity of the substrate and/or by adjusting the biomolecule concentration in the network.

In an aspect, the substrate comprises a prestrained polymeric material.

In an aspect, the prestrained polymeric material comprises polystyrene and/or polyvinyl chloride.

In an aspect, the method is a one pot process.

In accordance with an aspect, there is provided an antimicrobial material, a microarray, a wearable patch, a smart textile, a wearable device, a flexible electronic, an adhesive/repellent surface, a biosensor, a bioassays, a cell culture substrate, a biomimetic material, a surface-enhanced Raman spectroscopy, an electrode, a catalyst, a drug delivery device, or an energy storage material comprising the wrinkled network of claim 1.

In accordance with an aspect, there is provided a method to shrink pre-strained material, the method comprising:
a) Using pre-strained materials of any shape and curvature, flat or 3D as substrates.
b) Optionally, depositing another material intended to form wrinkled morphologies onto the substrates.
c) Exposing the substrates with or without a deposited material to a high-pressure gas.

Wherein the high-pressure gas induces the shrinkage of the pre-strained substrates.

In an embodiment, the pre-strained substrate comprises but is not limited to prestressed polystyrene sheets, prestressed polyvinyl chloride sheets.

In an embodiment, the high-pressure gas comprises a gas which functions as a plasticizer, optionally, carbon dioxide or hydrogen.

In an embodiment, the shrinkage of pre-strained substrates when another material is deposited onto the substrates results in the deposited material crimping internally and folding to form diverse wrinkled morphologies.

In an embodiment, the wrinkling process preserves the sophisticated micro/nanostructure of the deposited materials.

In an embodiment, the material depositing method comprises but is not limited to inkjet printing, 3D printing, physical/chemical vaporous deposition, electrospinning, drop casting, and spin coating.

In an embodiment, the deposited materials can be deposited in controllable sizes or patterns, comprising but not limited to films, dot arrays, line arrays, and 3D structures, including 3D printed material and material produced via both reductive manufacturing and additive manufacturing.

In an embodiment, the deposited material intended to form wrinkled morphologies comprises biological or non-biological (synthetic) materials.

In an embodiment, the biological or non-biological (synthetic) materials comprise heat or solvent sensitive or insensitive materials including but not limited to one or more of proteins, peptides, nucleic acids, viruses, bacteriophages (phages), and polymers.

In an embodiment, the deposited bacteriophages (phages) comprise but are not limited to natural phages and engineered phages, and phage derivatives.

In an embodiment, the phage depositions present a 4-level hierarchical structure, wherein the phage nanofilaments self-assemble into orderly-aligned submicron bundles, which crimp into tunable microscale wrinkles on size-controllable micro-arrays.

In an embodiment, the wrinkle morphologies of the deposited material can be tuned by one of more of: i) controlling the thickness of the deposited material, ii) surface engineering of the substrate, including but not limited to controlling the shrinkage ratio of the substrate, iii) changing the concentration of the deposited material.

In an embodiment, the deposited material with 3D wrinkled morphologies can be used as a scaffold to deposit other material, comprising but not limited to nanoparticles, deoxyribozymes, cells, or thin film metal coatings.

In an embodiment, the deposited mixture with 3D wrinkled morphologies can be used in applications comprising biosensing, drug-loading/delivery, bioassays, cell culture substrates, biomimetic materials, surface-enhanced Raman spectroscopy, new electrodes, catalysts, and energy storage materials.

In an embodiment, the deposited mixture can be used in applications comprising new electrodes, catalysts, and energy storage materials.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### BRIEF DESCRIPTION OF THE DRAWINGS

Certain embodiments of the disclosure will now be described in greater detail with reference to the attached drawings in which:

FIG. 1. Inkjet printing of phage bioink on polystyrene substrates. a. Schematic of high-throughput printing of phage bioinks and crosslinking chemistry leading to formation of ordered array of phage gel microdots. b. Pictures of a flexible chip with phage bioink microarrays, visible as white dots. c. Fluorescence images of air died phage gel microdot made with fluorescein-tagged phages (scale bar: 100 μm) and a higher magnification area showing 2D reticular texture (scale bar: 10 μm). d. Top: Reconstructed 3D confocal image of the phage gel microdot from panel c. Bottom: Confocal Z-stack images of the cross section of microdots printed on untreated and plasma-treated substrates respectively. Scale bar: 100 □m. e. Photos of phage gel microdot arrays on a plasma-treated substrate before and after immersing in water with shaking at 270 rpm for 24 hrs. Scale bar: 5 mm. f. Rheological characterization of phage bioink before and after gelation. Left: Storage modulus (G′) and loss modulus (G″) of the bioink during amplitude sweeps with oscillation strain ranging from 1% to 100%. Right: Frequency sweep curves for the bioink with frequencies ranging from 0.1 Hz to 10 Hz with 10% strain. g. Size control of the phage gel microdots. Top: Wetting behaviours of a water droplet on an untreated polystyrene substrate (left) and a treated polystyrene substrate (right). Middle: Bright field images of phage microdots printed on corresponding substrate. Scale bar: 500 μm. Bottom: Diameter distribution of phage microdots printed on corresponding substrates at different bioink volume for each dot. n=16 microdots from 4 independent experiments per group. Box plots showed minimum to maximum (whiskers), 25-75% (box), median (band inside) with all data points. Statistical significance was derived from one-way Analysis of Variance (ANOVA) with Tukey's multiple comparison test. ****p-value <0.0001.

FIG. 2. The crosslinking reactions between M13 phage nanofilaments (length: 880 nm, width: 6.6 nm) and EDC.

FIG. 3. Stable attachment of phage gel microdot arrays on the substrates. a. Scanning photos of phage gel microdot arrays (gelled) attaching on the untreated and plasma-treated substrates before and after shaking in water for 24 hrs. Arrows indicate the locations of eluted phage gel microdots after washing. b. Scanning photos of fresh-printed phage bioink microdots (not gelled) were easily washed off from the prestrained polystyrene immediately after immersing into the water (˜2 s). Scale bar: 5 mm.

FIG. 4. Tunable flower-shaped wrinkled patterns on phage gel microdots obtained through high pressure carbon dioxide (HPCD) method. a. Schematic of the shrinkage of prestrained polystyrene substrate (PPS) in HPCD environment (79 bar, 35° C.) driving the crimp of attached phage gel microdots, where different dot thicknesses resulted in different wrinkled morphologies. b-e. Left: Pictures of the phage gel microarrays with and without HPCD method. Right: SEM images of phage gel microdot array, a single microdot, and the microstructure of the dot. The microdots were made under different conditions: b. Untreated substrate, without HPCD method. c. Untreated substrate, with HPCD method. d. Treated substrate, with HPCD method. e. Treated substrate; phage gel microdots were pre-aired before HPCD process. f. Left: schematic of the definition on microdot diameter and distance. Middle: Diameter of microdots prepared with different preparation conditions. (n=9, 15 microdots in 3 independent experiments for untreated and treated groups, respectively). Right: Distance between two adjacent microdots on x axis and y axis. n=16 and 12 measurements in 3 independent experiments for untreated and treated groups, respectively. Box plots show minimum to maximum (whiskers), 25-75% (box), median (band inside) with all data points. Statistical significance was derived from one-way ANOVA with Tukey's multiple comparison test. ****p-value <0.0001.

FIG. 5. HPCD did not change the morphology of phage gel microdots on unshrinkable substrates. a-b. Pictures of phage microarrays (13 nL of bioink per dot) on a glass slide a. before and b. after HPCD treatment. c-d. SEM images of a phage gel microdot c. before and d. after HPCD treatment. e-f. Zoomed-in SEM images of the phage gel microdots in panel c-d, respectively.

FIG. 6. a. SEM image of a phage gel microdot on plasma-treated substrate made with 13 nL of bioink. b. Zoomed-in SEM image of the phage gel microdot in panel a. c. Further zoomed-in SEM image of the phage gel microdot in panel a showing the nanofibrous texture on the phage gel microdot surface.

FIG. 7. Quantitative analysis of wrinkle width. a-c. Contrast-enhanced SEM images of wrinkled microstructures corresponding to the wrinkle morphologies in FIG. 4c-e, respectively. d. Zoomed-in image of panel c. Inset arrows indicate the width of wrinkles.

FIG. 8. Width distribution of the wrinkles at the central area of phage gel microdots. (n=50 measurements in 5 independent microchips for untreated group. n=52 measurements in 5 independent microchips for treated groups.) Violin plot lines indicate 25^{th}, 50^{th}, 75^{th} percentile with all data points. Statistical significance was derived from one-way Analysis of Variance (ANOVA). ****p<0.0001, and p=0.1867 between two treated groups.

FIG. 9. Morphology difference at the edge areas of the wrinkled phage gel microdots obtained under different conditions. a-c, SEM images of the edge areas of the wrinkled phage gel microdots.

FIG. 10. Width distribution of wrinkles at the edge areas. Phage gel microdots on untreated substrate are not included here because of the unclear borders of the wrinkles at the edge area. (n=20 measurements in 5 independent microchips for HPCD group. n=25 measurements in 5 independent microchips for air-dry+HPCD groups.) Violin plot lines indicated 25^{th}, 50^{th}, 75^{th} percentile with all data points. Statistical significance was derived from unpaired t-test. ****p<0.0001.

FIG. 11. Effect of bioink volume on wrinkled morphology. SEM images of phage gel microdots (3.25 nL and 13 nL of bioink respectively) on untreated substrates a. before and b. after HPCD shrinkage.

FIG. 12. Quantitative data of wrinkled phage gel microdots on untreated PPS substrates while using 3.25 nL of bioink volume for each dot instead of 13 nL. a. Distance between two adjacent phage gel microdots with and without HPCD shrinkage. Compare to data for 13 nL size phage gel microdot shown in FIG. 4f. b. Diameter of phage gel microdots with and without HPCD shrinkage. Compare to data for 13 nL size phage gel microdot shown in FIG. 4f. c. Wrinkle width at the central area of the phage gel microdots. Compare to data for 13 nL size phage gel microdot shown in FIG. 8. (n=9 independent phage gel microdots per group in panel a and b. n=25 measurements in 5 independent microchips in panel c.) Box plots showed minimum to maximum (whiskers), 25-75% (box), median (band inside) with all data points. Violin plot lines indicated 25^{th}, 50^{th}, 75^{th} percentile with all data points. Statistical significance was derived from unpaired t-test. ****p<0.0001.

FIG. 13. Effect of the concentration of phage ink on the wrinkle morphology. From left to right: SEM images of the phage gel microdot array, one single dot, the microstructure in the center of the dot and the microstructure on the edge of the dot. Inserts are the partial enlarged pictures of corresponding images. The phage gel microdots were printed on untreated substrates, and the phage concentrations were a. 5.0×10¹³ PFU mL⁻¹ and b. 2.5×10¹³ PFU mL⁻¹.

FIG. 14. Heat shrinkage of prestrained polystyrene sheets under 140° C. for 10 mins. a. Photos of three sizes of polystyrene sheets before and after heat shrinkage under 140° C. Three sizes of the films are 35 mm×25 mm, 25 mm×20 mm, and 15 mm×10 mm respectively. b. Size of the prestrained polystyrene sheets before and after heat shrinkage in panel a showing isotropic and size-independent shrinkage ratio (n=3 independent sheets per size group). Data were presented as mean±SD with all data points. Statistical significance was derived from unpaired t-test. ****p<0.0001.

FIG. 15. Heat shrinkage of substrate at 145° C. for 10 mins creates wrinkles on phage gel microdots. SEM images of a. the array of heat shrunk phage gel microdots on a prestrained polystyrene substrate, b. a single shrunk phage gel microdot and c-d. the wrinkle structure on the phage gel microdot surface.

FIG. 16. Shrinkage mechanism of prestrained polystyrene (PPS) films through high pressure carbon dioxide (HPCD) method (79 bar, 35° C.). a. Schematic of HPCD shrinkage mechanism. b. Left: Transmittance intensity at different rotation angles of PPS before and after HPCD shrinkage under polarized optical microscopy. n=3 independent experiments per condition. Data were presented as mean±SD. Right: Polarized optical microscopy images of PPS at 0° and 45° before and after HPCD shrinkage, respectively. c. Glass transition temperature of the prestrained polystyrene sheets. The dotted vertical line and arrows indicate the position of T_{g}. d. SEM images of PPS surface before and after HPCD shrinkage. e. Photos of three sizes of PPS before and after HPCD shrinkage. Three sizes were 35 mm×25 mm, 25 mm×20 mm, and 15 mm×10 mm. f. Size change of the three sizes of sheets in panel e. n=9 independent films per group. Data were presented as mean±SD with all data points. Statistical significance was derived from paired t-test. ****p-value <0.0001.

FIG. 17. Strain-stress curves of prestrained polystyrene substrates before and after HPCD shrinkage. Tensile strength assessment as a function of material position, with the slope of a stress/strain curve indicating the Young's modulus.

FIG. 18. Disappearance of the birefringence of prestrained polystyrene substrates after shrinkage, indicating the reducing orientation of polymer chains. Polarized optical microscopy images at different rotation angles of a prestrained polystyrene film a. before shrinkage, b. after HPCD shrinkage, and c. after heat shrinkage.

FIG. 19. Cross-sectional SEM images of prestrained polystyrene substrates before and after HPCD shrinkage.

FIG. 20. Shrinkage was not observed in non-prestrained polystyrene sheets after HPCD treatment. a. Photos of a prestrained polystyrene sheet before shrinkage, after heat shrinkage (releasing prestrain), and after heat shrinkage followed by HPCD treatment. b. Size change of the three phases of the prestrained polystyrene sheet in panel a (n=3 independent sheets). Data were presented as mean±SD with all data points. Statistical significance was derived from two-way ANOVA with Tukey's multiple comparison test. ****p<0.0001. ns (not significant) p=0.9185, 0.7154 and 0.9983 for the group of length, width, and thickness, respectively.

FIG. 21. Shrinkage was not observed in prestrained polystyrene sheets subject to atmospheric pressure CO₂. a. Photos of a prestrained polystyrene sheet at 0 h and 72 h in low-pressure CO₂ (1 bar, 35° C.). b. Size change of the three phases of the prestrained polystyrene sheet in panel a (n=3 independent sheets). Data were presented as mean±SD with all data points. Statistical significance was derived from paired t-test. ns (not significant) p=0.9283, 0.9899 and 0.9999 for the group of length, width, and thickness, respectively.

FIG. 22. Shrinkage was not observed in prestrained polystyrene sheets in dry ice which provided a drastic temperature change. a. Photos of a prestrained polystyrene sheet at 0 h and 72 h exposed to low temperature CO₂ gas (dry ice, 1 bar, −78.5° C.). b. Size change of the three phases of the prestrained polystyrene sheet in panel a (n=3 independent sheets). Data were presented as mean±SD with all data points. Statistical significance was derived from paired t-test. ns (not significant) p=0.9862, 0.9862 and 0.9999 for the group of length, width, and thickness, respectively.

FIG. 23. Anisotropic shrinkage of polyvinyl chloride (PVC) through the HPCD treatment. a. Photos of a prestrained PVC sheet before and after HPCD shrinkage. b. Size change of the three phases of the prestrained PVC sheet in panel a (n=3 independent sheets). Data were presented as mean±SD with all data points. Statistical significance was derived from paired t-test. ****p<0.0001. ns (not significant) p=0.9998.

FIG. 24. Four-level hierarchical structures of phage gel microarrays after high pressure carbon dioxide (HPCD) treatment. a. Schematic of the four-level hierarchical structures on phage-built microarrays from microscale to nanoscale. b. Morphologies of the microdot surface before and after the formation of microscale wrinkles. Left: 3D atomic force microscopy (AFM) images of the microdot surface before and after height of the microdots. Right: Surface roughness profile of the microdot surface with and without HPCD method. c. Submicron bundles aligning orderly on the microwrinkles. Left: SEM images of the bundles (coated with 3-nm Pt). Right: Width distribution of the nanobundles. n=26 measurements in 4 independent experiments. Violin plot lines indicated 25^{th}, 50^{th}, 75^{th} percentile with all data points. d. Self-assembled phages nanofilaments composing the bundles. 1. AFM images of the microwrinkles. 2. AFM images of the structure on the bundles. 3. Schematic of the phage alignment according to the AFM image. 4. Surface roughness profile of the bundles along the arrow direction in part 2.

FIG. 25. Large-scale Phage films exhibiting wrinkled microstructure through HPCD method. SEM images with varying levels of magnification showing the hierarchical surface on a phage hydrogel film on treated polystyrene substrates after HPCD method.

FIG. 26. Bovine serum albumin (BSA) microdots exhibiting wrinkle microstructures via HPCD method. From left to right: SEM images of BSA microarray, single BSA microdot and the microstructures on the dot a. without and b. with HPCD method. BSA microdots were prepared with 2% BSA and 0.1 M EDC.

FIG. 27. Wrinkled phage gel microdots maintaining same microstructure after immersing in water with shaking for 72 hrs. Bright field microscopy images of the wrinkled phage gel microdots before immersing in water, and after immersing in water in the shaking incubator (37° C., 270 rpm) for 10 mins, 12 hrs, and 72 hrs.

FIG. 28. Phage-built microscaffolds loaded with biorecognition molecules for biosensing applications. a. Schematic illustration of the process for evaluating the structural stability of phage microscaffolds loaded with biotinylated antibody interleukin 6 before and after HPCD shrinkage. b. Left: Fluorescence images of flat and wrinkled phage-antibody microarrays after incubating with streptavidin-cy5. Right: Fluorescence intensity of flat and wrinkled phage-antibody microarrays relative to flat and wrinkled phage-exclusive microarrays (control) after incubating with streptavidin-cy5. n=12 microdots in 3 independent experiments per group. ****p-value <0.0001, and p=0.5244 between two controls. c. Schematic illustration of the process for applying phage-RFD microarrays for Legionella pneumophila detection. RFDs: aminated RNA-cleaving fluorescent DNAzymes. d. SEM images of a phage-RFD microdot and higher magnification view showing surface morphology. e. Left: Reconstructed 3D confocal image of a phage-RFD microarray. Right: Fluorescence intensity profile of the six microdots in the array across the z-axis. f. Initial fluorescence intensity of flat and wrinkled phage-RFD microarrays relative to flat, two-dimensional RFD arrays. n=4 independent experiments per group. g. Decrease ratio of fluorescence signal in flat/wrinkled phage-RFD and phage gel microarrays after washing, i.e., incubating in the buffer solution for 24 hrs. n=8 independent microarrays for the wrinkled group, and n=4 independent microarrays for each of the other two groups. h. Decrease ratio of fluorescence signal of washed flat and wrinkled phage-RFD microarrays after 24-hr incubation with buffer solution (control) and extracellular solution extracted from L. pneumophila culture solution at different titer. n=12 microdots in 3 independent experiments per group. **p-value=0.0035 and 0.0040 (wrinkled microdots). ns (not significant) p-value=0.0752 and 0.8899 (flat microdots) for the group of 10⁶ and 10⁵ CFU mL⁻¹, respectively. i. L. pneumophila test of real-world liquid samples of contaminated water (sample 1: culture+, sample 2: culture+, sample 3: culture-) collected from cooling towers, using wrinkled phage-RFD microdot arrays. n=12 microdots in 3 independent experiments per group. ****p-value (between sample 1 or 2 and the wrinkled control group in panel h) <0.0001. ns (not significant) p-value (between sample 3 and the wrinkled control group in panel h) >0.999. p-value <0.05 between a test sample and a control (bacteria-free water) is considered L. pneumophila-positive. Box plots showed minimum to maximum (whiskers), 25-75% (box), median (band inside) with all data points. Bar chats were presented as mean±SD. Statistical significance was derived from two-way ANOVA with Tukey's multiple comparison test.

FIG. 29. Stability of DNAzyme added to phage gel microdots through the shrinkage process. a. Fluorescence images of phage+DNAzyme microdots before and after washing and shrinkage. b. Quantified fluorescence intensity of phage+DNAzyme microdots in panel a. (n=14 independent microdots for the group of treated substrate before shrinkage; n=16 independent microdots per group for the other three groups.) Box plots showed minimum to maximum (whiskers), 25-75% (box), median (band inside) with all data points. Statistical significance as derived from paired t-test. ****p<0.0001.

FIG. 30. Fluorescent images and signal distribution curves of fluorescin-tagged phage microdots before and after HPCD process. Blue channel: microdots excited at 340 nm and imaged using: blue channel a λ=435 nm optical filter; Orange channel: microdots excited at 528 nm and imaged using a λ=590 nm optical filter; Green channel: excited at 465 nm and imaged using a λ=515 nm optical filter. The fluorescent intensity of microdots were characterized along the corresponding arrows in the images under green channel.

### DETAILED DESCRIPTION

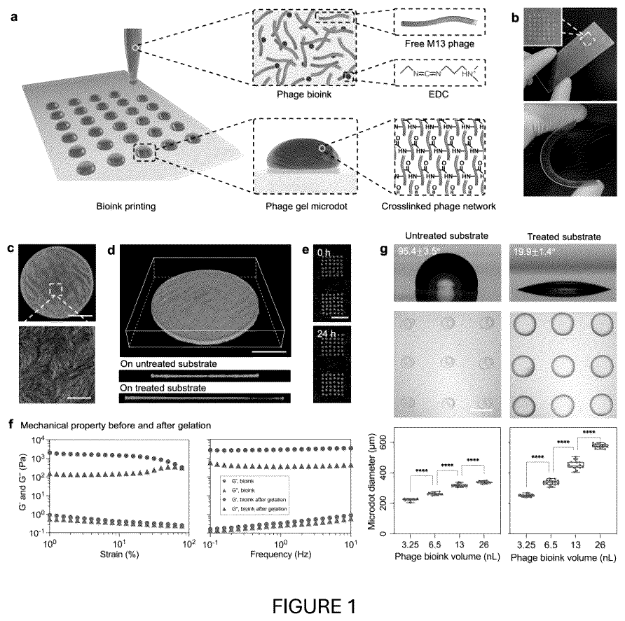

In aspects, the present disclosure provides phage microdots with unique 3D hierarchical wrinkle morphologies by a temperature and solvent independent substrate-shrinkage technology. A phage bioink was used to print soft phage microarrays with controllable size on prestressed polystyrene substrates (FIG. 1a). Subsequent substrate shrinkage triggered by high pressure CO₂ induced wrinkled morphologies on the attached phage microdots while preserving their sophisticated nanostructures, a result that could not be achieved with heat or solvent-based shrinkage. As the co-product of phage self-assembly and the shrinking process, phage microdots presented a unique four-level hierarchical micro/nanostructure with adjustable patterns from “sunflower” to homogenous wrinkles, demonstrating a highly-porous biointerface with high specific surface area. This hierarchical phage-built network was applied as a microscaffold to design functional wrinkled structures with DNAzymes for sensing pathogenic strains of Legionella Pneumophila collected from contaminated facilities.

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included may be explicitly excluded by way of proviso or negative limitation, such as any specific compounds or method steps, whether implicitly or explicitly defined.

### II. Products

Described herein are molecular networks. In particular, wrinkled networks based on biomolecules are described. Furthermore, wrinkled networks based on heat- or solvent-sensitive materials are also described herein. While wrinkled networks made of synthetic materials are known, biomolecule-based wrinkled networks are new and made by novel methods that do not require the use of heat or solvents. In this way, heat- or solvent-sensitive biomolecules can be used to form wrinkled networks, alone or in combination with synthetic materials. Such methods typically involve shrinking prestrained supports upon which a biomolecule network is disposed.

Hierarchically structured materials, such as wrinkled structures, are nature's way of packing a very high surface area into a small footprint. The existing methods for making artificial wrinkled structures, however, are designed for polymers and metals and thus inherently incompatible with biomolecules. Most such methods are reliant on high temperatures and/or organic solvents and are harsh and disruptive towards biologics such as proteins, nucleic acids, or viruses.

In certain aspects, the wrinkled network is nano-reticular. In additional or alternative aspects, the wrinkled network comprises a hierarchical architecture, with two, three, four or more levels of hierarchy. For example, when the wrinkled network is formed from phage, it typically comprises four layers of hierarchy: phage nanofilaments, which self-assemble into orderly-aligned submicron bundles, which crimp into tunable microscale wrinkles, which are formed on size-controllable micro-arrays.

Advantageously, the wrinkled network described herein is tunable, meaning its pattern of wrinkle shape and wrinkle density are adjustable as desired to meet certain parameters. For example, adjusting the hydrophobicity of the substrate and/or adjusting the biomolecule concentration in the network will lead to different wrinkle shapes and densities.

Typically, the biomolecule is a phage. However, it will be understood that any biomolecule, including combinations of biomolecules, may form the basis of the wrinkled networks described herein. Advantageously, the methods described herein do not require the use of heat or solvents and, thus, biomolecules can be used without comprising their structure or utility. For example, the biomolecule in aspects comprises a protein, a nucleic acid, a bacteria, a virus, a phage, or any combination thereof.

It will be understood that the networks described herein may contain various additives and/or impurities. For example, the networks may include components that are the result of the preparation methods involved. For example, when phages are used as the biomolecule, other materials may be included in the network that may be considered impurities or may be added as beneficial excipients. Examples include proteins/peptides, enzymes, polymers, metals, salts, or ions. Furthermore, although the networks are based on a biomolecule, they may include various non-biological or synthetic components.

In certain aspects, the wrinkled network includes a cargo. The cargo may be heterogeneously or homogeneously distributed throughout the wrinkled network. Typically, the cargo is homogenously distributed throughout the wrinkled network. Various cargos may be included depending upon the desired end use of the wrinkled network. As a particular example, the cargo may comprise a biorecognition element for sensing the presence of a particular cell of interest, such as a white blood cell or a specific bacterial cell, for sensing the presence of an analyte of interest, for sensing the presence of genetic material, specific proteins, enzymes, and so on. The analyte could be in blood or a component thereof, sweat, on skin, in a water source, a food source, and on an on. Advantageously, the networks described herein could be used to test for almost any analyte or cell with a specific signature that is detectable. In certain aspects, the biorecognition element detects an enzyme, antibody, antigen, nucleic acid, cell, aptamer, tissue, microorganism, organelle, cell receptor, or any combination thereof.

In aspects, the biorecognition element is, thus, an antibody, an enzyme, an aptamer, a cell, a nucleic acid, a protein, or any combination thereof. In particular examples, the enzyme comprises a deoxyribozyme.

The wrinkled networks described herein may be configured in a number of different ways. In some aspects, the networks are configured in an antimicrobial material, a microarray, a wearable patch, a smart textile, a wearable device, a flexible electronic, an adhesive/repellent surface, a biosensor, a bioassays, a cell culture substrate, a biomimetic material, a surface-enhanced Raman spectroscopy, an electrode, a catalyst, a drug delivery device, or an energy storage material.

Thus also provided herein is an antimicrobial material, a microarray, a wearable patch, a smart textile, a wearable, a flexible electronic, an adhesive/repellent surface, a biosensor, a bioassays, a cell culture substrate, a biomimetic material, a surface-enhanced Raman spectroscopy, an electrode, a catalyst, a drug delivery device, or an energy storage material comprising the wrinkled networks described herein.

### III. Methods

Also described herein is a universal method to induce wrinkled structures in various biomolecule-based materials, including virus-built hierarchical scaffolds, while preserving their nanoarchitecture. This is a transformative method, free of heat and solvents, that allowed for the preservation of micro and nanostructure of biologics while inducing substrate wrinkling. A phage bioink was used to print soft phage microarrays with controllable size on prestressed polystyrene substrates. Subsequent substrate shrinkage induced 2D phage microarrays to fold into complex 3D flower-like structures. The functional phage microarray was demonstrated for its ability to detect Legionella bacteria from industrial water samples.

Advantageously, the methods described herein a heat-free, using a maximum temperature of about 35 degrees Celsius, which is friendly to heat-sensitive materials. Further, dehydration and wrinkling can be achieved at the same time, which is particularly advantageous for hydrogels. The methods preserve the sophisticated molecular, nano, and micro structures during wrinkling. Further, diverse wrinkle morphology easily achievable by controlling the parameters of the method.

In aspects, described herein is a method for wrinkling a biomolecule-based network disposed on a substrate. The method typically comprises shrinking the substrate without using heat or solvent.

Typically, shrinking the substrate comprises applying a high pressure plasticizer gas to the substrate. Examples of a plasticizer gas include carbon dioxide and/or hydrogen.

As described above, the biomolecule typically comprises a protein, a nucleic acid, a bacteria, a virus, a phage, or any combination thereof.

Generally, the methods comprise printing the biomolecule-based network on the substrate and further comprise crosslinking the biomolecule-based network prior to shrinking the substrate. In this way, the network is firmly adhered to the substrate before the substrate is subject to shrinking. Further, the network is typically dried substantially or fully before shrinking the substrate.

As described herein, the wrinkle shape, density, and/or homogeneity are tunable by adjusting the hydrophobicity of the substrate and/or by adjusting the biomolecule concentration in the network.

Typically, the substrate comprises a prestrained polymeric material. Examples of a prestrained material in polystyrene and/or polyvinyl chloride.

Advantageously, the method described herein is typically a one pot process. This means that all components in the network can be mixed together and applied at once, resulting in improved homogeneity.

### EXAMPLES

The following non-limiting examples are illustrative of the present disclosure:

### Example 1: Virus-Assembled Biofunctional Microarrays with Hierarchical 3D Nano-Reticular Network

Three dimensional (3D) hierarchical wrinkled materials built with biological entities have so far remained exclusive to nature. We create multiscale functional ultraporous 3D bio-networks of bioprinted phage-built wrinkled microarrays through establishing a universal heat- and solvent-independent substrate-shrinkage method induced by high-pressure carbon dioxide (HPCD). This method results in diverse wrinkled patterns on soft materials and is particularly powerful for solvent- and heat-sensitive biomaterials, for which other methods have failed. The phage nanofilaments (7 nm width) self-assemble into orderly-aligned submicron bundles (100 nm width), which crimp into tunable microscale wrinkles (0.7-5.0 μm width) on size-controllable micro-arrays (200-600 μm width) exhibiting a four-level hierarchical nano-reticular structure. The HPCD method also protects the bioactivity of biorecognition molecules loaded into the microarrays, leading to the design of bacteria-sensing chips, made with in-house deoxyribozyme-loaded 3D phage microarrays. The developed bacteria-sensing chips achieve a limit of detection that was 100× more sensitive with greater reproducibility compared to two-dimensional microdot arrays and correctly identify Legionella pneumophila in contaminated water samples collected from industrial cooling towers, highlighting phage-built wrinkled networks as a platform for bottom-up assembly of biological building blocks into biofunctional material.

### 1. Introduction

Inspired by nature, hierarchically-structured biomimetic materials have been revolutionizing fields ranging from tissue engineering¹⁻³ to biosensing^{4,5} to (bio)fouling mitigation^{6,7}. Wrinkled materials are among the most versatile hierarchical structures and have far-reaching applications in wearables⁸, flexible electronics⁹, energy conversion¹⁰, and adhesive/repellent surfaces^{11,12}. In nature, wrinkled structures are made with proteins as the foundational components that drive the structural hierarchy and add nanoarchitecture to the material. Likewise, these foundational components are responsible for the unique functionalities of wrinkled structures that would otherwise be limited without them. Such added capabilities include rapid environmental trigger-response, as is the case of white blood cells¹³, or contractility, as is the case of esophageal mucosa¹⁴. Nature has so far remained the exclusive engineer of such sophisticated structures with biomimicry efforts hindered by the lack of compatible technologies and reliable methodologies that can preserve the nanoarchitecture of proteins and proteinaceous bionanoparticle building blocks. One of the most common methods of inducing wrinkles is utilizing the mismatching deformation in a bilayer system composed of a shrinkable substrate and the attached material. These properties are commonly triggered by mechanical stretching¹⁵, heat^{12,16-18}, and/or solvents^{19,20}. The heat-induced substrate-shrinkage and solvent-induced swelling-shrinkage can efficiently create wrinkle patterns. In doing so, however, these methods are known to denature proteins and proteinaceous bionanoparticles, thereby preventing the desired nanoarchitecture²¹. The substrate stretching/release method is only applicable to very specific substrate-coating combinations, and is commonly accompanied by solvent or heat that cannot be readily integrated into refined microprinting systems¹⁵.

Viral nanoparticles such as bacteriophages (bacterial viruses) are attractive candidates for introducing the missing nanoarchitecture within engineered wrinkled structures. This is largely due to their properties that are unmatched by engineered nanoparticles, namely monodisperse self-replication, remarkable diversity in shape and size, and precise control of surface chemistry through chemical and/or genetic modification^{22,23}. When leveraged together, these properties make bacteriophages powerful natural building blocks for the next generation of biomimetic designs²⁴⁻²⁶ Previous work has demonstrated that nanofilamentous phages are capable of self-organizing at high concentrations, forming 2-dimensional (2D) films²⁷, bulk hydrogels²⁸⁻³⁰, and spheres^{31,32} leading to micro and macro scale materials with powerful optical and structural properties. In total, viral-based material platforms represent the next frontier for creating functional 3D structural materials. Despite the enormous promise, a universal method to induce wrinkled structures in various biomolecule-based materials, including virus-build hierarchical scaffolds, while preserving their nanoarchitecture, remains elusive.

To address this unmet technological need, we developed a heat- and solvent-independent substrate-shrinkage approach that allowed us to create phage microdots with unique 3D multiscale wrinkle morphology. A phage bioink was used to print soft phage microarrays with controllable size on prestressed polystyrene substrates (FIG. 1a). Subsequent substrate shrinkage triggered by high pressure CO₂ induced wrinkled morphologies on the printed phage microdots while preserving their sophisticated nanostructures; a result that could not be achieved using existing heat or solvent-based shrinkage methods. As the co-product of phage self-assembly and the shrinking process, phage microdots presented with a unique four-level multiscale micro/nano-architecture with adjustable patterns from sunflower-like to homogenous wrinkles. This demonstrates a highly porous biointerface with high specific surface area. The hierarchical phage-built network was then used as a microscaffold to design functional wrinkled structures with in-house designed deoxyribozymes (DNAzymes) for detecting pathogenic strains of Legionella pneumophila in contaminated water samples from cooling towers.

### 2. Results and Discussion

2.1. High-Throughput Printing of Phage-Built Microdot Arrays with Nanoreticular Texture

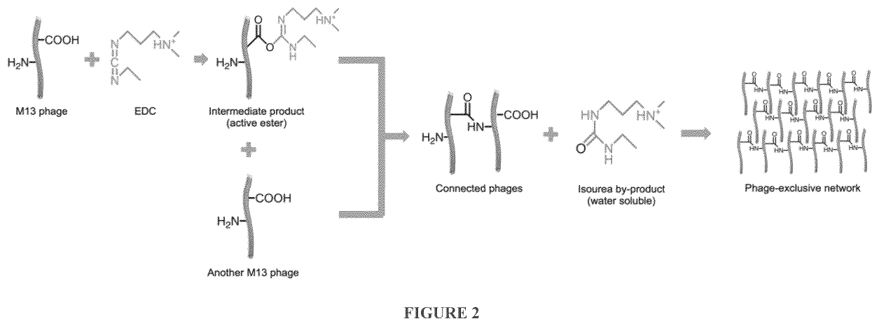

We developed a high-throughput strategy to fabricate ordered arrays of phage gel microdots onto shrinkable polystyrene substrates. As illustrated in FIG. 1a, we constructed a bioink consisting of M13 nanofilamentous phages (10¹⁴ PFU mL⁻¹) as the sole building blocks and 1-Ethyl-3-(3-dimethylaminopropyl) carbodiimide (EDC, 0.1 M) as a zero-length crosslinker. An inkjet printer applied a nanoliter droplet (as low as 0.65 nL) of bioink onto a substrate, creating precise microdot arrays. The polystyrene substrate at this stage was a flexible chip with visible ordered arrays of phage bioink (FIG. 1b). The substrate was then transferred to a sealed humid environment, where the bioink microdots gelled in situ and solidified into phage gel microarrays. Each gel microdot represented an independent phage-exclusive network where nanofilamentous phages were crosslinked through amide bonds¹ (FIG. 2). At this stage, the phage gel microdots exhibited 2D reticular texture (FIG. 1c) and the air-dried gels were flat round discs, regardless the hydrophilicity of substrates (FIG. 1d). ¹ Crosslinking reaction between M13 phages and EDC. 1-Ethyl-3-(3-dimethylaminopropyl) carbodiimide (EDC) has been used as a carboxyl activating agent to induce the intermolecular coupling of carboxyl groups with amine groups¹. M13 phages exhibit abundant carboxyl and amine groups on their protein capsid, which EDC can crosslink. EDC has been previously used to stabilize phages as a two-dimensional film on flat rigid substrates². Here, we mixed phage and EDC as a bioink and based on the chemical equation (FIG. 2), we expect none of the atoms from EDC (labelled in red) to be coupled into the built phage network. EDC first reacted with the carboxyl groups on the phage capsid to form an intermediate product with an active ester group that later reacted with the amine groups. Consequently, the phages were connected by amide bonds and formed phage hydrogels/microgels. In other words, EDC took oxygen atoms from carboxyl groups on phages and formed a water-soluble isourea by-product which can be easily washed out. Thus, a phage-exclusive network is achieved using simple M13+EDC bioink.

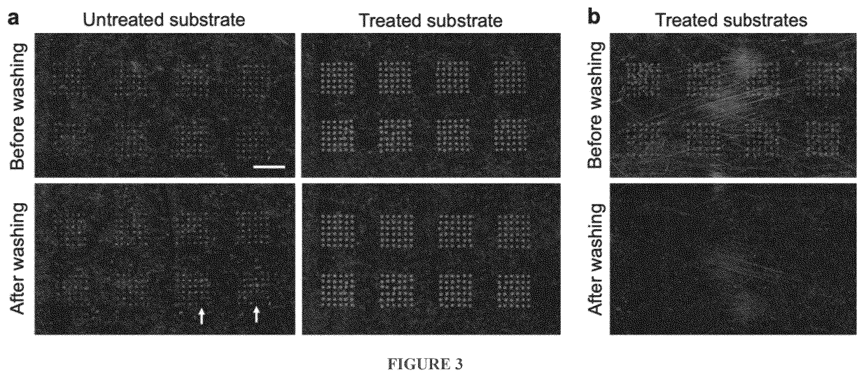

We further confirmed that the phage gel microdots attached firmly to the substrates, which is an important for wrinkling through substrate shrinkage. The phage gel microdots remained on the original printed location after being immersed in water with shaking for 24 hrs (FIG. 1e). Notably, phage gel microdots printed on plasma treated substrates attached more firmly (FIG. 3a), whereas fresh-printed phage bioink was washed off immediately (FIG. 3b). The gelation of phage bioink not only helped irreversibly attach phages to the substrate, but it also improved the mechanical properties of the microdots, which was verified through rheological characterization (FIG. 1f). At 12 hrs post deposition, the ink behaved as a gel, which has a distinctly higher storage modulus (G′) than loss modulus (G″) compared with the original phase (before gelation).

The diameter of phage gel microdots was controlled through adjusting bioink volume and substrate hydrophilicity (FIG. 1g). The original polystyrene substrate had a water contact angle of 95.4±3.5°. By dosing the bioink volume for each dot between 3.25 nL and 26 nL, the diameter of the microdots varied from 222.5±7.8 μm to 338.8±6.0 μm. The bioink volume can be further increased to achieve a broader size range. In addition to controlling the bioink volume, plasma treatment on substrates reduced the contact angle of the polystyrene substrate significantly (to 19.9±1.4°), increasing the hydrophilicity of the substrate. Consequently, the same volume of bioink spread covered a larger footprint on the substrate, further broadening the range of the microdot diameter (between 251.5±7.7 μm and 577.65±13.95 μm). Hydrophilicity treatment adds to our ability to fine tune the size of the microdots, and as will be explained below, was also observed to affect the wrinkling morphology.

### 2.2. Formation of Tunable Flower-Shaped Patterns on Phage Microarrays Via High-Pressure Carbon Dioxide Method

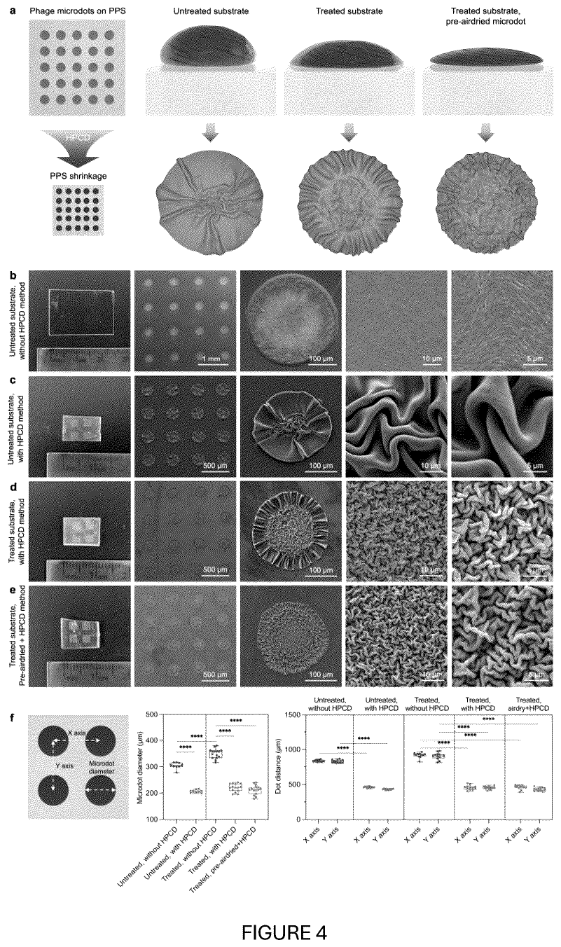

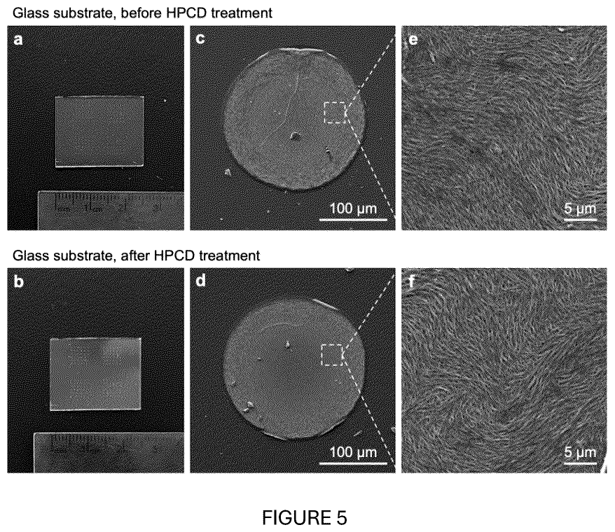

Avoiding high-temperature environments and organic solvents is important for applying shrinkage methods to phage-built materials. We realized wrinkling can be achieved by immersing the printed phage gel microarrays into high-pressure carbon dioxide (HPCD) at a protein-friendly temperature (≤physiological temperature 37° C.) (FIG. 4a). The surface of the original phage gel microdots (13 nL bioink per dot) was flat at the microscale, where ordered and aligned phage bundles at sub-microscale were observed (FIG. 4b). In HPCD environment (79 bar, 35° C.), the homogeneous shrinkage of prestrained polystyrene (PPS) substrates drove these attached phage gel microdots to crimp internally and fold. Consequently, all phage gel microdots going through HPCD method exhibited consistent flower-shaped folds (FIG. 4c). The central area of each microdot was highly folded while the edge area only had several radial wrinkles. The fibrous texture was clearly visible, both in the center and on the radial wrinkles. It is noteworthy that phage gel microdots printed on unshrinkable glass substrates did not exhibit wrinkled morphologies through the same process (FIG. 5), indicating that the wrinkles were caused by the designed substrate shrinkage, not any other factor in the HPCD method.

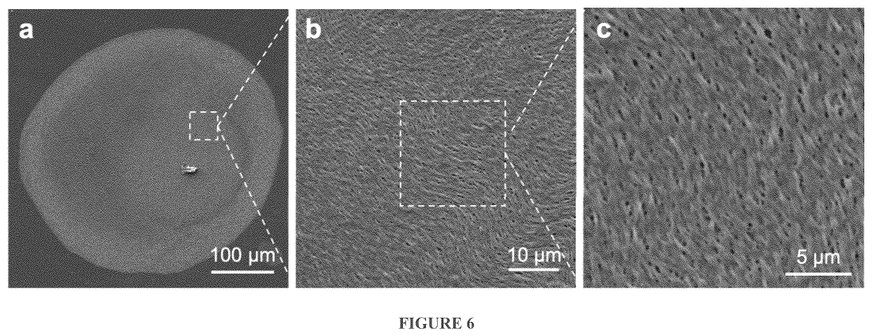

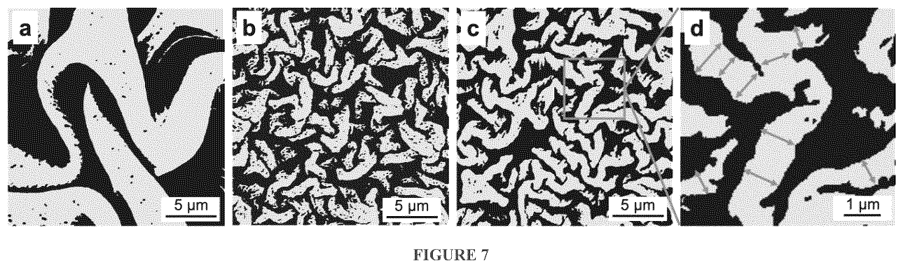

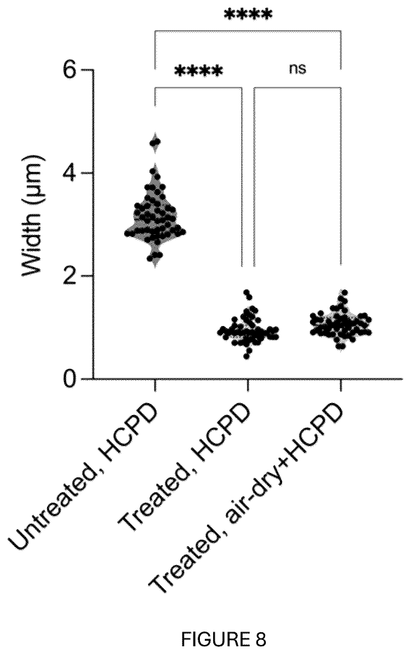

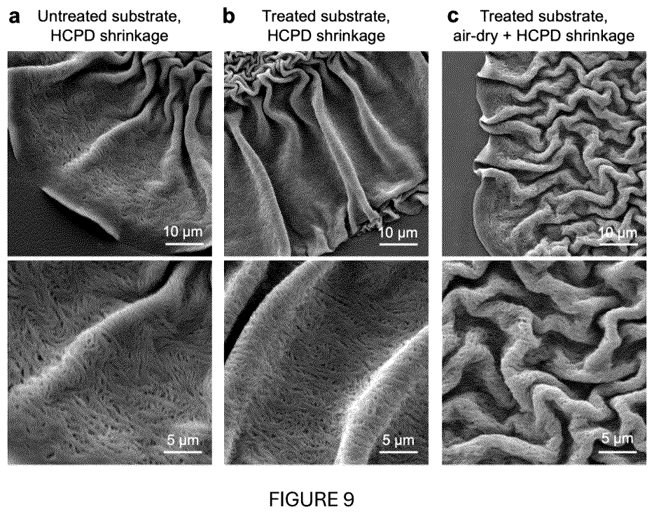

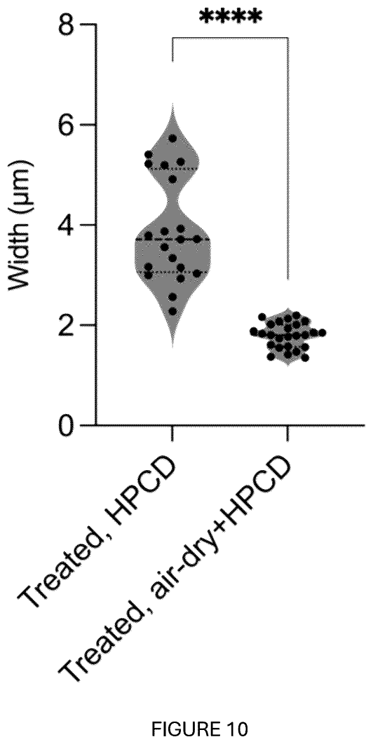

To achieve a more homogenous morphology that could be advantageous for biosensing applications, the flower-shaped pattern on phage gel microdots was diversified by adjusting microdot thickness. As shown in FIG. 1g, hydrophilicity treatment of PPS substrates led to the spreading of the bioink, increasing its footprint while keeping the volume the same. This resulted in the formation of larger phage microdots (indicating a decreased thickness) with a similar submicron-bundle morphology comparing to the phage gel microdots on non-treated substrates (FIG. 1g and FIG. 6). The reduction in microdot thickness facilitated the formation of internal folds during substrate shrinkage, increasing the size of the region with compact folds. The improvement of microdot attachment on treated substrates (FIG. 3a) is expected to have increased the shear force on the attached microdot further contributing to increase in the region with compact wrinkles. Consequently, the obtained pattern for these microdots through the same HPCD method was distinct compared to those observed on untreated substrates (FIG. 4d). The wrinkles at the central area of the microdot were narrow and curly, while the edge had wider and radial wrinkles (packed closer than radial wrinkles on untreated substrate), resembling a “sunflower” structure. Furthermore, air-drying the microdots before the HPCD process further decreased the microdot thickness and consequently generated the most homogenous wrinkled morphology (FIG. 4e). Quantitatively, the wrinkle width at the central area of phage gel microdots decreased significantly from 3.16±0.48 μm for untreated substrate to 0.95±0.23 μm after switching to treated substrates (FIGS. 7 and 8). Pre-airdrying did not change the central wrinkle morphology significantly (1.06±0.22 μm). Instead, the effect of pre-airdrying showed up on the morphology at the edge area of microdots (FIG. 9), reducing the wrinkle width from 3.89±1.03 μm to 1.79±0.25 μm (FIG. 10). We further verified isotropic wrinkling by quantifying the microdot diameter and the distance between adjacent microdots after substrate shrinkage (FIG. 4f). The decrease in phage gel microdot diameter through HPCD was 32.0% for microdots on untreated substrates, and 37.0% for microdots on treated substrates. This led wrinkled microdots on treated and untreated PPS to attain a similar size distribution at the end of the shrinkage process. The different diameter decrease ratio can be attributed to tighter attachment of phage gel microdots to plasma treated PPS substrates, causing these microdots to experience a higher shear force at the microdot-PPS interface during substrate shrinkage. The consistent distance reduction between phage gel microdots along x and y directions clearly illustrated that the polystyrene shrinkage through HPCD method was isotropic.

In summary, the surface morphology of phage gel microdots (wrinkling diameter and wrinkling pattern) was tuned through adjusting the hydrophobicity of the substrate and pre-airdrying of the phage gel microdots, representing three different flower-shaped micropatterns, where the wrinkle width varied from 5.0 μm to 0.7 μm. These diverse morphologies with controllable wrinkle width provided robust range of microscaffold morphology and may be used to optimize performance of biorecognition elements with different shapes, sizes, and conformations.

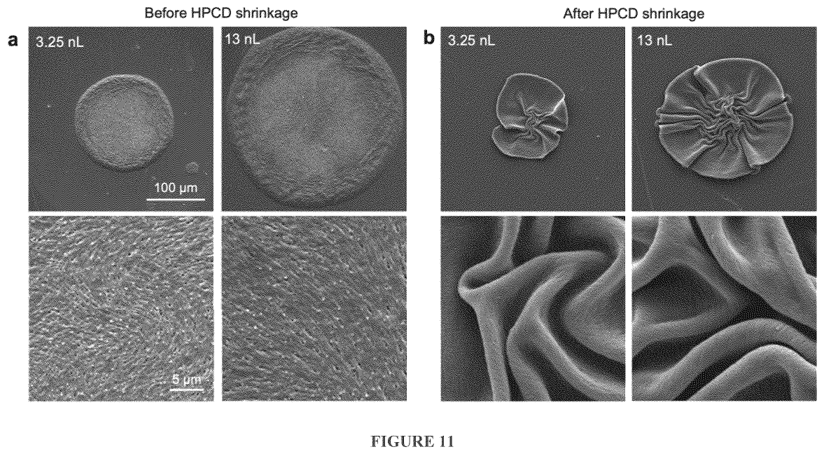

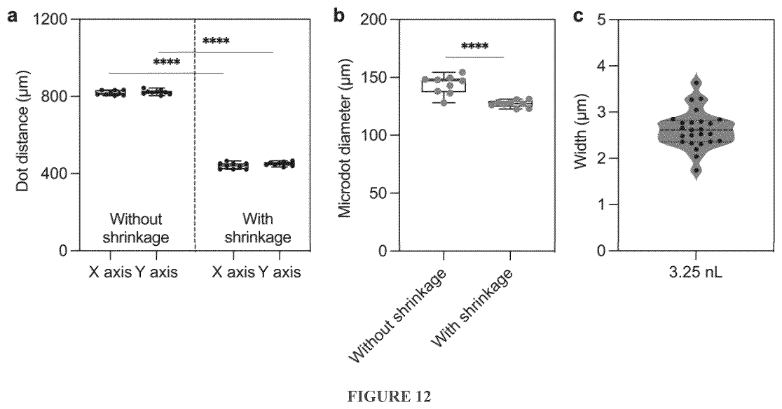

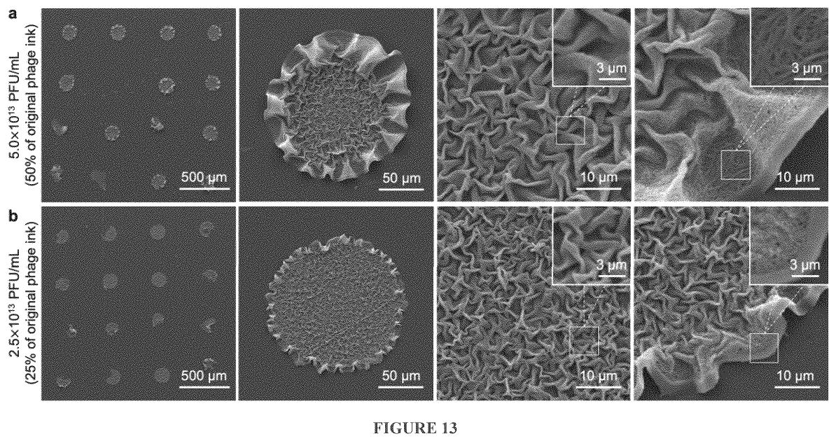

In addition to substrate surface pre-treatment, two other parameters were found to affect wrinkle morphology, namely microdot volume and phage concentration. Phage gel microdots made with 3.25 nL droplets had similar wrinkle morphology as microdots made with 13 nL under the same condition (FIG. 11). The wrinkle width was not significantly reduced (from 3.16±0.48 μm to 2.63±0.41 μm), indicating that changing bioink volume alone could not effectively manipulate the wrinkle structure or morphology (FIG. 12). Concurrently, we found that reducing phage concentration resulted in wrinkles to be narrower and more homogeneous. As shown in FIG. 13a, reducing phage concentration by half (to 5×10¹³ PFU mL⁻¹) resulted in finer wrinkles and the expansion of central wrinkled area. This phenomenon was even more pronounced when the concentration was further reduced to 2.5×10¹³ PFU mL⁻¹ (FIG. 13b). It should be noted that the phage gel microdots were too soft at low phage concentrations, causing the deformation and breakage of some phage gel microdots, leading us to conclude that hydrophilicity control of the substrates was the more efficient and potentially scalable method to tune the wrinkling morphology.

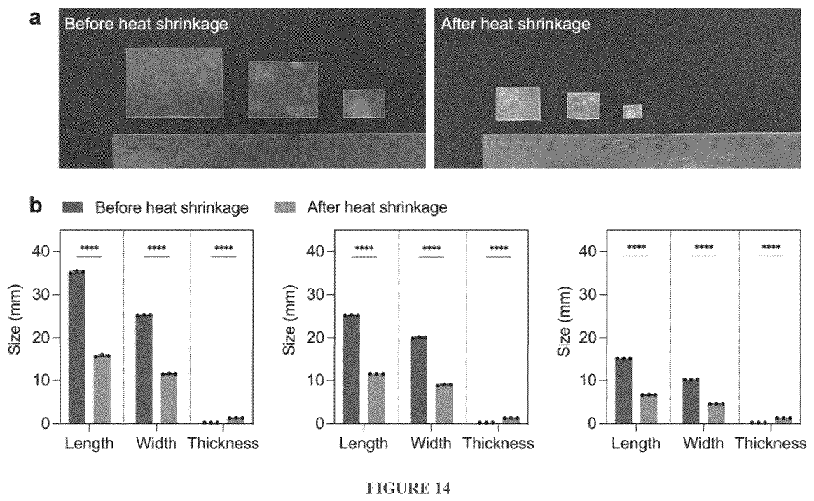

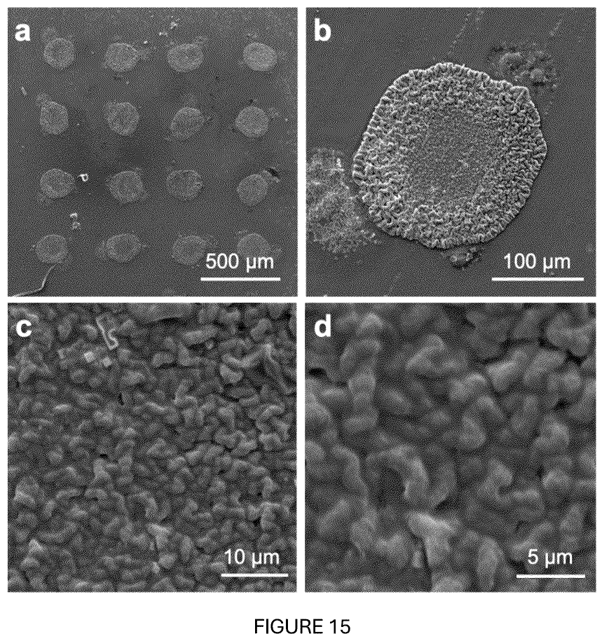

In one of the most widely adopted methods of wrinkling, PPS shrinkage is triggered by heating above substrate glass transition temperature (T_{g}≈102.6° C.), as reported in the literature^{12,16} and verified in FIG. 14. We demonstrated that heat shrinkage indeed created wrinkled morphologies on the attached phage gel microdots (FIG. 15). However, the wrinkled pattern can no longer be described as a nano-reticular network because the fibrous micro/nano architecture is almost completely destroyed. This observation proved that heat shrinkage failed to preserve the delicate and sophisticated nanostructures on phage gel microdots. It further highlights the core advantage of HPCD method, namely being free of heat exposure, which makes it an ideal approach to create wrinkled morphologies on materials made with or containing biological entities or molecules sensitive to high temperature, such as proteins, peptides, antibodies, enzymes, and viruses/bacteriophages, thus preserving their sophisticated micro/nano architecture.

### 2.3. Shrinkage Mechanism in High Pressure Carbon Dioxide Environment for Prestrained Polystyrene Substrates

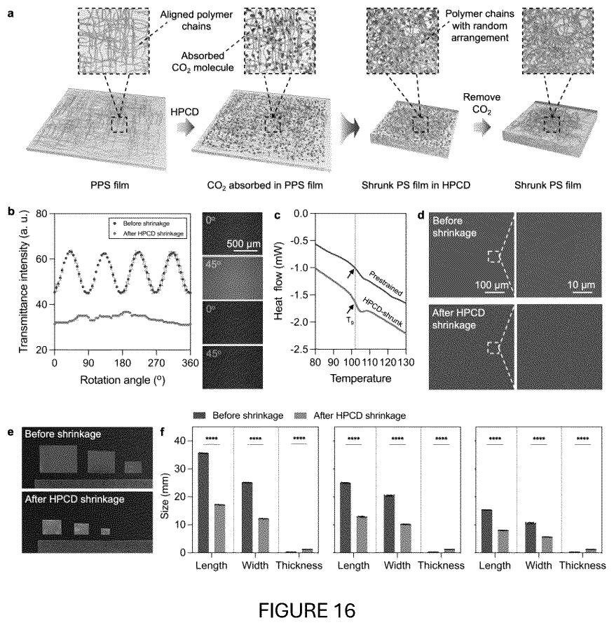

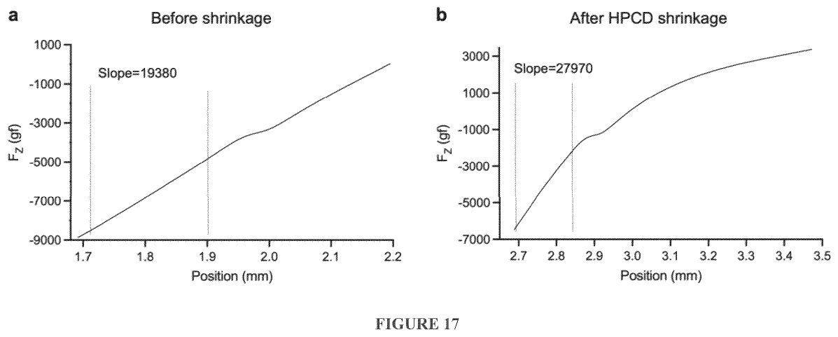

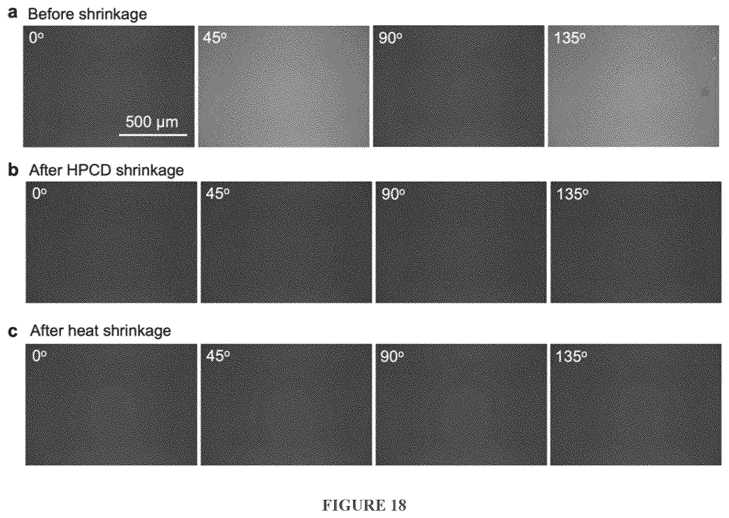

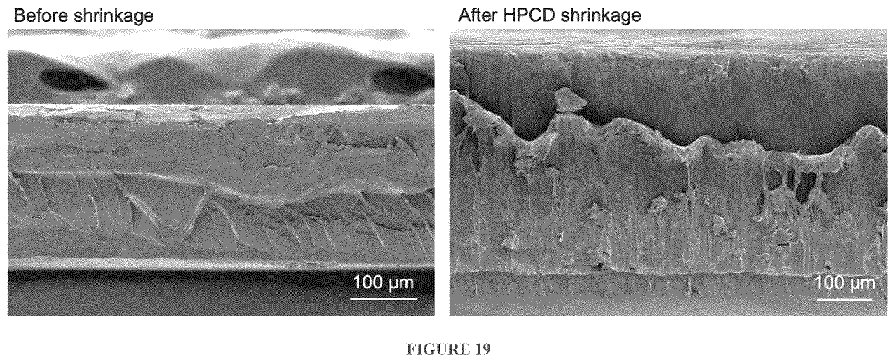

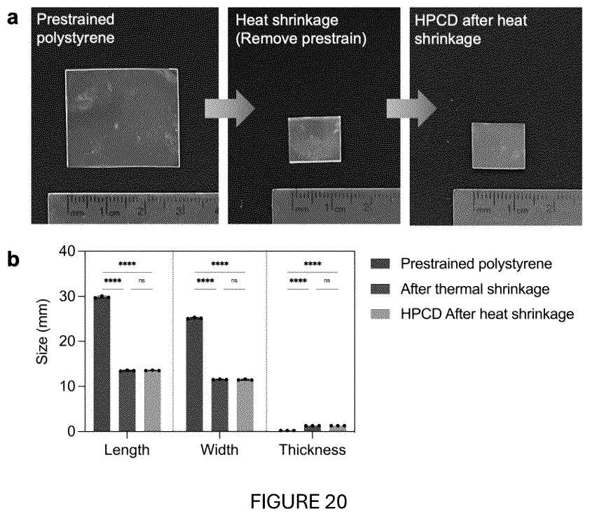

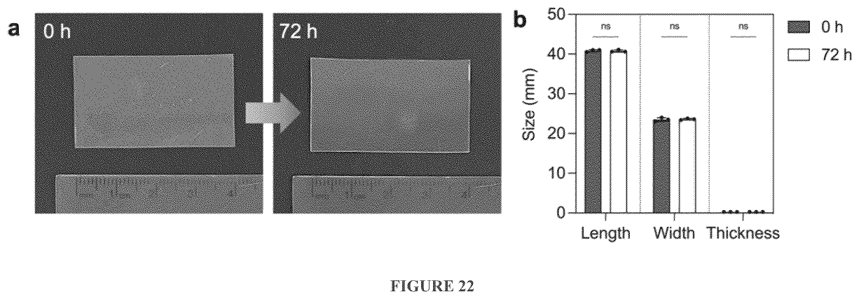

Motivated by the performance of HPCD method in creating tunable wrinkles in phage gel microdots, we further investigated the mechanism behind the shrinkage behavior of PPS in an environment of high-pressure CO₂. Carbon dioxide at high pressure has been reported as a plasticizer of regular polystyrene and some other polymers where the absorbed CO₂ molecules can increase free volume and polymer segment mobility^{33,34}, allowing chains to move freely past one another and resulting in the drastic decrease of glass transition temperature (T_{g}) according to the Chow model (equation (1))^{35,36}

$$ln ( \frac{{T}_{g , HPCD}}{{T}_{g , atm}} ) = β \cdot [ θ \cdot ln θ + ( 1 - θ ) \cdot ln ( 1 - θ ) ] \qquad ( 1 )$$

Where T_{g,HPCD} and T_{g,atm} are the glass transition temperatures of polystyrene under high pressure CO₂ and at atmospheric pressure, respectively; β and θ are the nondimensional parameters². It has been reported that compared to regular polystyrene, polymer chains for PPS are more aligned (resulting in birefringence) and spread apart in the pre-stretched directions³⁷. As illustrated in FIG. 16a, for 2-dimensional PPS pre-stretched in both x and y directions, we hypothesized that in the HPCD environment (79 bar), the reduction of T_{g} allowed these polymer chains to reduce back to the more random arrangement at 35° C., which led to shrinkage of PPS along the pre-stretched directions. The reduced alignment of polymer chains was verified through the disappearance of birefringence (FIG. 16b) and increased Young's modulus (FIG. 17). A similar loss of birefringence was observed after conventional heat shrinkage of PPS (FIG. 18). It is worth noting that after CO₂ being removed, T_{g} of shrunk polystyrene reverted back to the same as the original PPS (FIG. 16c). The polystyrene surface remained flat after shrinkage (FIG. 16d), and the cross-sectional morphology showed no significant change while the film became thicker (FIG. 19), which suggests that polymer chains were homogenously mobilized on both the surface and throughout the entirety of the PPS material. We observed consistent and isotropic shrinkage of PPS where the length and width both decreased 49.9±1.7% (FIG. 16e-f), independent of the size of the PPS. Prestraining of PPS, HPCD and mild temperature increase above T_{g,p} were all important in this method as no shrinkage was observed when any of these conditions was not met (FIG. 20-22). ² Shrinkage mechanism of prestrained polystyrene in high-pressure carbon dioxide (HPCD). In general, the glass transition temperature (T_{g}) of a polymer increases when pressure increases³. This is due to the reduced free volume and increased intermolecular interactions between polymer chains. However, in the case of polystyrene (PS) and other amorphous polymers, the T_{g} can be significantly affected by the presence of HPCD^{4,5}. When CO₂ is dissolved in PS, it acts as a plasticizer, reducing the T_{g} of PS. The mechanism behind this effect is the disruption of the intermolecular interactions between polymer chains by the presence of CO₂ molecules, which act as a diluent and increases the free volume of the polymer⁴. As pressure increases, more CO₂ molecules dissolve in PS, leading to a greater reduction in T_{g}.

The negative correlation of T_{g} and CO₂ pressure is described in the Chow model (equation (1))^{6,7}

$$ln ( \frac{{T}_{g , HPCD}}{{T}_{g , atm}} ) = β \cdot [ θ \cdot ln θ + ( 1 - θ ) \cdot ln ( 1 - θ ) ] \qquad ( 1 )$$

where T_{g,HPCD} and T_{g,atm} are the glass transition temperatures of polystyrene in HPCD and at atmospheric pressure, respectively; β and θ are the nondimensional parameters. It has been reported that T_{g} of PS decreased from 100° C. to approximately 40° C. when the surrounding CO₂ pressure reached 60 bar⁷. Consequently, HPCD has been used for PS foaming, extrusion, and additive incorporation into PS⁸⁻¹⁰.

Based on this mechanism, we applied HPCD on prestrained polystyrene (PPS) where polymer chains were stretched and the material was under tension (FIG. 3a). When HPCD was introduced to the PPS, it dissolved into the polymer matrix and created a plasticizing effect which caused the polymer chains to relax and contract, resulting in a reduction in the overall size of the material. The pressure of the CO₂ also compressed the material, further contributing to the shrinkage. It is important to note that the extent of shrinkage depends on the initial degree of pre-strain and the pressure and temperature conditions of HPCD treatment. Therefore, shrinkage was not observed for these three conditions:
1) un-prestrained PS sheets under HPCD (FIG. 18);
2) PPS sheets under low-pressure CO₂ (FIG. 19);
3) PPS sheets within dry ice which provided a drastic temperature change (FIG. 20).

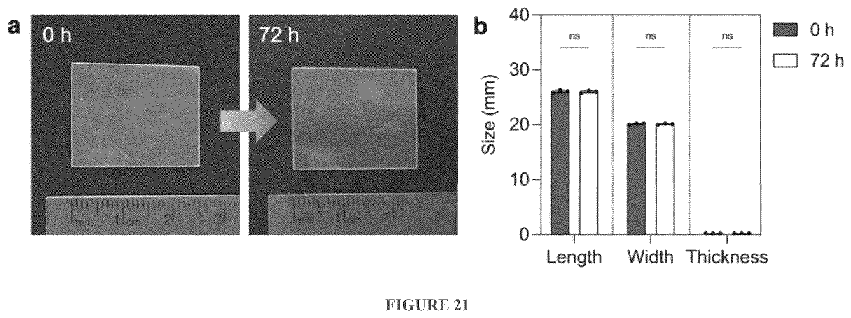

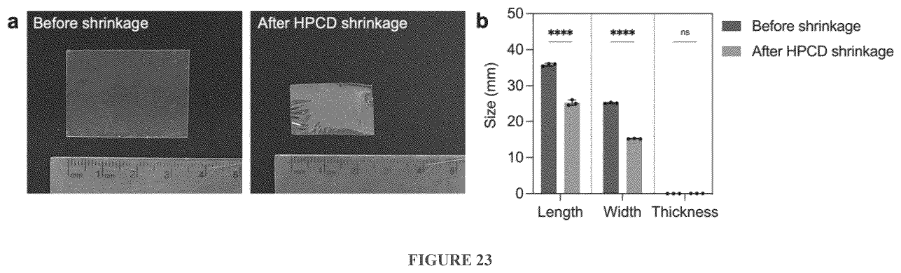

We further confirmed that HPCD method could be extended to other shrinkable materials, demonstrated by inducing anisotropic shrinkage of prestrained polyvinyl chloride, which was pre-stretched in one direction³ (FIG. 23). This observation further reinforces the hypothesized mechanism of HPCD shrinkage, suggesting that HPCD method is suitable for diverse prestrained materials and can potentially create more series of wrinkled patterns according to the prestrain direction/ratio of the substrates. ³ Anisotropic shrinkage of prestrained polyvinyl chloride. A prestrained polyvinyl chloride (PVC) film was pre-stretched to differing ratios in two independent directions. After HPCD treatment, the PVC film showed the anisotropic shrinkage phenomenon (FIG. 21). Quantitatively, the size of PVC film decreased 39.6% at one direction, while the size at the other direction only dropped 29.5%.

### 2.4. Unique Four-Level Hierarchical Structure of Wrinkled Phage Gel Microarrays

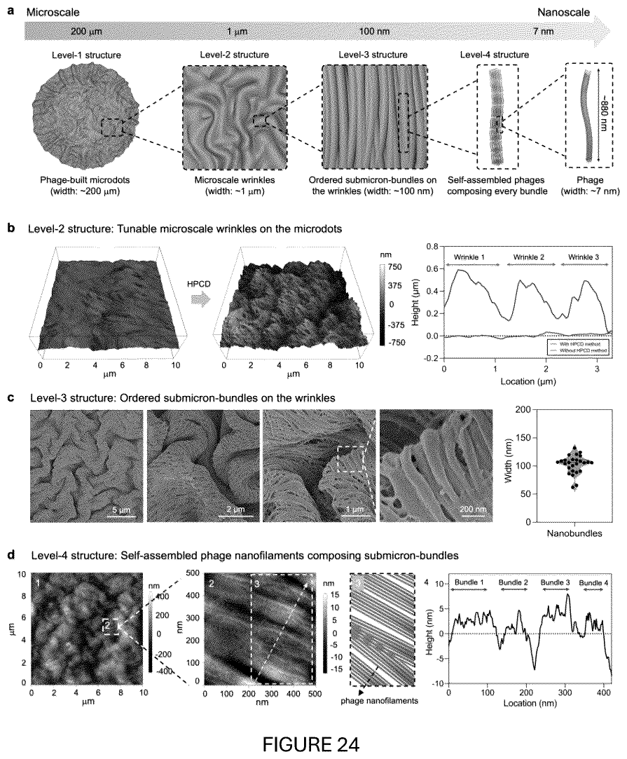

To confirm that HPCD preserved the nanoarchitecture of the 3D phage gel microarrays, we explored the submicron-bundle texture of the microscale wrinkles on phage gel microarrays. As described above, orderly-aligned and closely-adjacent bundles were apparent on the printed phage gel microdots before substrate shrinkage (FIG. 4b and FIG. 5-6). The electron micrographs in FIG. 4 suggested that nanofilament alignment and the phage bundles were preserved through the process of wrinkle formation. This led us to hypothesize that we had in fact created four levels of hierarchy in the wrinkled phage gel microdot, as illustrated in FIG. 24a. To verify this hypothesis, we imaged and characterized each level of hierarchy.

The substrate shrinkage process gave rise to the second-level structure, namely the microscale wrinkles (width: ˜0.7-5.0 μm) (FIG. 24b), while the first level was the size-controllable phage gel microdots, as previously described (diameter: 200-600 μm) (FIG. 1g). High resolution electron micrographs of the microscale wrinkles showed ordered submicron-bundles (level-3) on each wrinkle (FIG. 24c). The width of submicron-bundles were 102.5±16.4 nm and were significantly higher than the width of individual phage nanofibers (width: 6.6 nm, length: 880 nm). The gaps between bundles increased (compared to flat phage gel microdots, FIG. 5) because of folding. This phenomenon resulted in a more porous nanostructure. It has been previously reported that phage nanofilaments are able to self-assemble and form cholesteric liquid crystalline structures at high concentrations (above 0.2 mg mL⁻¹)^{24,27}, explaining how the phages in our bioink (1×10¹⁴ PFU mL⁻¹, equivalent to ˜2.7 mg mL⁻¹) self-aligned and assembled into the observed bundles. Roughness analysis of high magnification atomic force micrographs confirmed the fourth level of hierarchy to be self-assembled phage nanofilaments (width: ˜7 nm) that formed ordered the observed bundles (FIG. 24d).

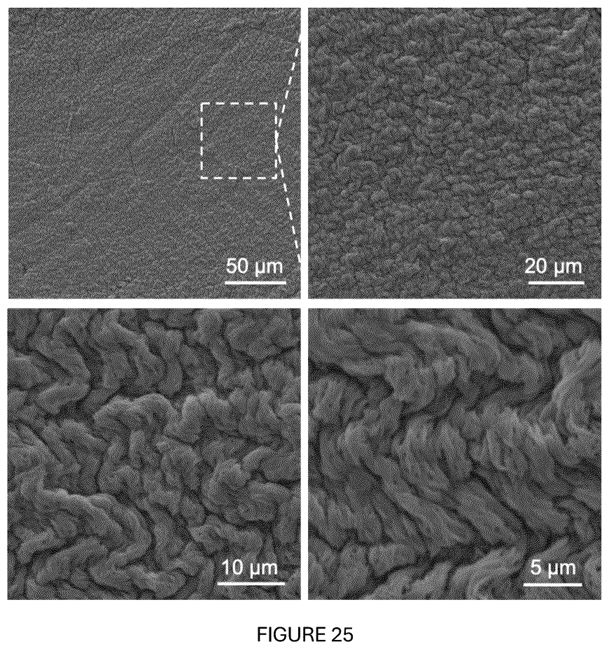

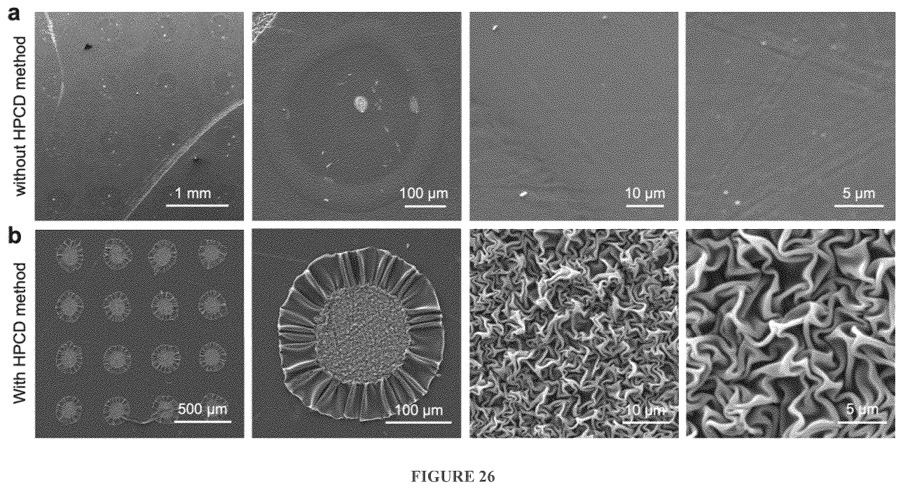

We further verified that the same four levels of hierarchy could be observed when macroscale phage films were processed by HPCD method. A phage hydrogel film over 4 cm² exhibited homogenous microwrinkles through same approach, and the nanofibrous textures on the wrinkles were well-preserved (FIG. 25). In addition, we verified that proteins could acquire a wrinkled microstructure via the HPCD method. As shown in FIG. 26, microdots made with 2% bovine serum albumin (BSA), crosslinked with EDC, formed similar “sunflower” morphology after HPCD process. However, the smooth surface of these wrinkles proved again that the additional levels of hierarchy of phage microdots (bundles and nanofibers) was a unique phenomenon generated by phage nanofilaments, not an artifact of the HPCD process.

In summary, our data supports the hypothesized unique 4-level hierarchical structure presented in FIG. 24a for phage gel microdots, which is not observed in control wrinkled structures without phage. This is strong verification that the microscale order imparted by liquid crystalline self-assembly of phages and their unique consistent nanofibrous architecture was preserved through the HPCD wrinkling method, while the heat-wrinkled phage gel microdots lacked anything beyond a 2-level hierarchy (FIG. 15). Our HPCD method thus proved to be a breakthrough in enabling the engineering of nature inspired levels of hierarchy using biological building blocks.

2.5. Biofunctional Phage-Built Microscaffolds with DNAzymes for Detecting Pathogenic Legionella pneumophila

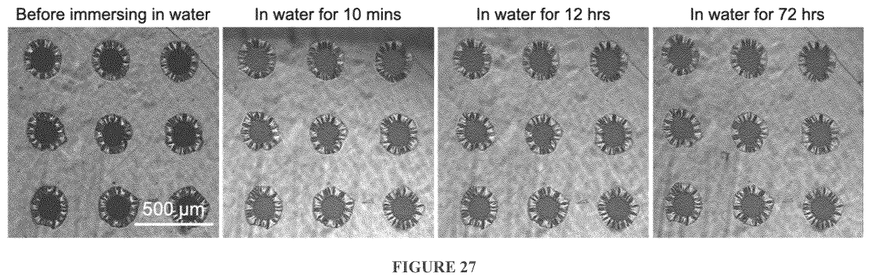

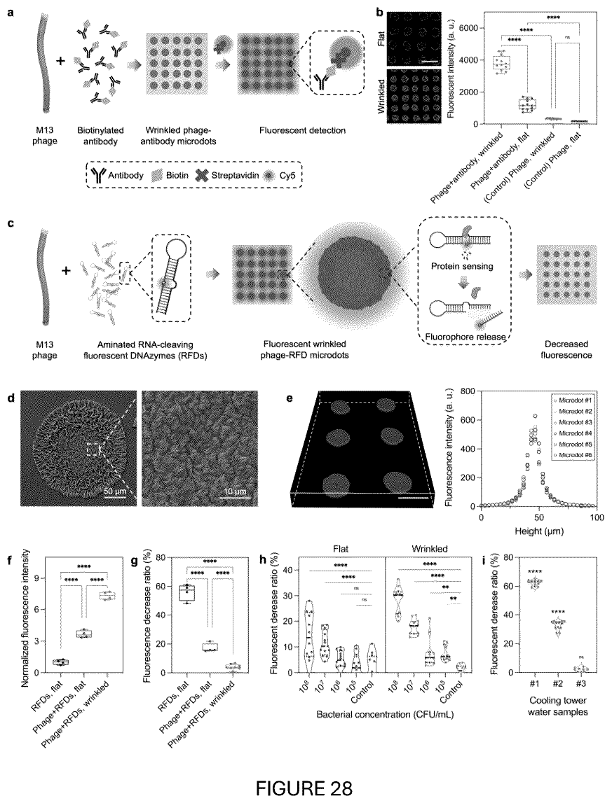

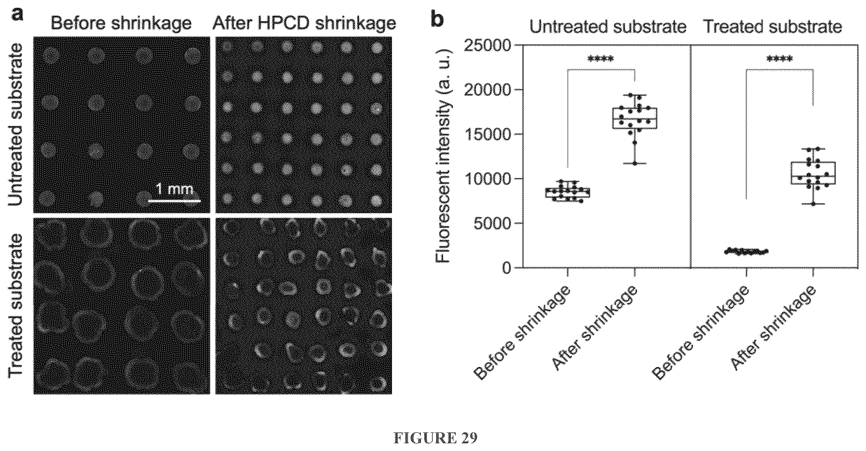

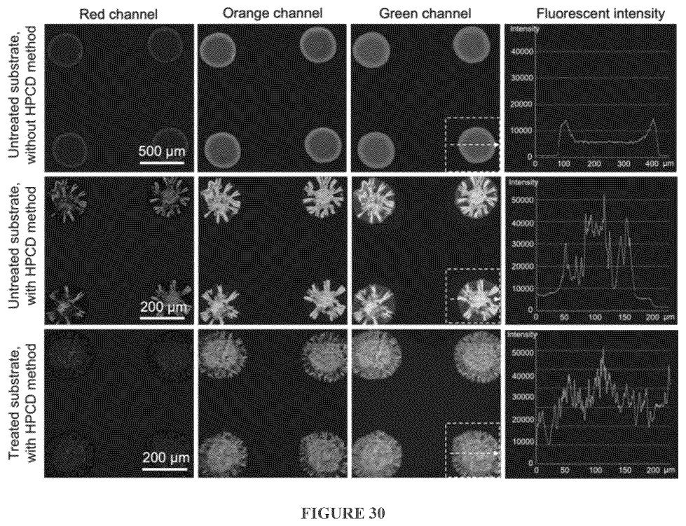

The developed 3D wrinkled phage gel microarrays enabled by the HPCD method offer greater levels of hierarchy than any other wrinkled structure, suggesting a higher surface area that could enhance the sensitivity of microarrays for biosensing compared to commonly used 2D arrays. Applying the phage-built microscaffolds for biosensing applications required two critical characteristics, namely structural stability, and the protection of loaded biorecognition elements (e.g., antibodies or DNAzymes). We observed long-term preservation of the wrinkled morphology of phage gel microdots even when exposed to long-term (72-hrs) shear forces under water with shaking (270 rpm) (FIG. 27). Loading of biorecognition molecules into the structural microscaffolds was achieved through printing a hybrid bioink (phages, crosslinker and biorecognition elements) followed by HPCD substrate shrinkage. We observed that the loaded antibodies (FIG. 28a-b⁴) and DNAzymes (FIG. 29) remained bioactive, proving that HPCD method did not affect the bioactivity of cargo in the phage gel microscaffolds. Interestingly, signal from the biorecognition elements was significantly increased after HPCD process due to the formation of 3D structures and increased surface density of fluorophores (FIG. 28a-b, FIG. 29-30). The wrinkled phage-DNAzyme microarrays on untreated substrates exhibited a strong fluorescence signal that was 95.1% higher than the flat microdots while this signal intensity increased 481.9% for the microarrays on treated substrates (FIG. 29). It can be concluded that in addition to enriching and protecting the sophisticated structures of phage gel microscaffold, HPCD method was able to preserve the biofunctionality of loaded biomolecules. ⁴ Structural preservation of antibodies during HPCD treatment. Anti-interleukin 6 (IL-6) antibodies have been widely used to treat certain inflammatory conditions, such as rheumatoid arthritis, juvenile idiopathic arthritis, and Castleman's disease¹¹⁻¹³. In this study, biotinylated IL-6 antibodies (IBA) were applied as a representative protein with functional structures to fabricate wrinkled composite phage-antibody microdots (FIG. 28a). Firstly, IBA was mixed with phage bioink and went through HPCD treatment after gelation. To verify whether HPCD treatment affected the functional structures of IBA, an enzyme-linked immunosorbent assay was performed, where the composite microdot chips were immersed in the solution of cy5-conjugated streptavidin that bound to IBA specifically. As shown in in FIG. 28b, the fluorescent intensities of the control samples (without IBA) were invisible compared to phage-IBA microdots (85.5% less), showing a minimal non-specific attachment of streptavidin-cy5 to phages. Notably, IBA remained stable and functional to bind to streptavidin throughout HPCD treatment. The wrinkled phage-IBA microdots exhibited a strong fluorescent signal that was 220.7% higher than the flat microdots. This phenomenon demonstrated the structural preservation of antibodies in the HPCD process and the signal-enhancement property of three-dimensional wrinkled morphology.

After confirming the preservation of biorecognition elements in the HPCD method, the biosensing efficacy of phage microscaffolds was evaluated through loading an in-house aminated RNA-cleaving fluorescent DNAzymes (RFDs) specific to Legionella pneumophila, a pathogenic bacterium commonly found in lakes, rivers, and cooling towers in heating, ventilation, and air conditioning systems. L. pneumophila is the most common cause of waterborne outbreaks worldwide and is associated with severe clinical risks, including respiratory failure, shock and acute kidney and multi-organ failure³⁸. To mitigate these clinical risks, and to ultimately save lives, a stable and biofriendly water-body detection tool is needed³⁸. The in-house L. pneumophila-specific RFDs were composed of an enzymatic and a fluorophore-labelled substrate strand, hybridized to one another³⁹⁻⁴². When exposed to L. pneumophila, the enzymatic strand interacted with a species-specific protein, subsequently inducing cleavage at a ribonucleotide site present on the substrate strand and yielding release of the fluorophore. The resultant decrease in fluorescence output signaled the presence of L. pneumophila⁴⁰. We hypothesised that loading RFDs into the phage microscaffold would integrate, protect, and significantly enhance signal compared to flat 2D microarrays, enabling rapid and direct detection of L. pneumophila from environmental samples without the need for slow and laborious culture-based methods (FIG. 28c).

As shown in FIG. 28d, the addition of RFDs did not interfere with the hierarchical structures of phage-built microscaffolds; RFDs were homogenously distributed throughout the microdot (FIG. 28e). To evaluate performance, we first assessed the original fluorescence signal on flat and wrinkled phage-RFD microdots relative to flat RFD arrays covalently printed onto planar glass substrates (FIG. 28f). Quantitively, the flat phage-RFD microdots exhibited a 264.9% increase in fluorescence intensity relative to the planar RFD arrays, partially supporting the claim that RFD were significantly integrated in the microscaffolds. Furthermore, HPCD shrinkage method concentrated the embedded RFD to a hierarchical 3D matrix with smaller diameter and greater depth, resulting in a further 101.2% increase in fluorescence signal over the flat phage-RFD microdots. To evaluate the immobilization stability of RFDs, the microarrays were washed with buffer solution for 24 hrs. As shown in FIG. 28g, over 56.0% of RFDs was eluted from planar RFD arrays. In contrast, the flat phage-RFD microdots only lost 17.2% signal and the wrinkled phage-RFD microdots further reduced this loss to only 4.0%. These results illustrate the significantly higher stable immobilization of RFDs in phage microscaffolds.

After confirming the hypothesized advantages of employing phage-built wrinkled gels as microscaffolds for the DNAzymes, we applied the wrinkled phage-RFD microarray as biosensors for L. pneumophila detection. The pre-washed microarrays were incubated in crude extracellular solution extracted from different concentrations of L. pneumophila cultures (10⁵ to 10⁸ CFU mL⁻¹) for 24 hrs. Flat phage microarrays were tested in parallel to evaluate the impact of the wrinkled structure on biosensing. As shown in FIG. 28h, the wrinkled phage microdot arrays offered a significantly greater decrease in fluorescence signal compared to their flat counterparts. Furthermore, wherein the flat microdots offered a limit of detection (LOD) of 10⁷ CFU mL⁻¹, the wrinkled phage microdots exhibited a LOD of 10⁵ CFU mL⁻¹. It is worth noting that wrinkled microdots yielded more consistent readouts as evidenced by the coefficient of variation (CV) data presented in Table 1.

TABLE 1
Coefficient of variation of the decrease ratio of fluorescence
signal of flat and wrinkled phage-RFD microarrays in FIG. 5h

| | 10⁸ | 10⁷ | 10⁶ | 10⁵ | Control |
|---|---|---|---|---|---|
| Wrinkle | 18.22% | 16.53% | 72.40% | 39.23% | 873193.73% |
| Flat | 65.50% | 43.76% | 63.04% | 412.61% | 1746749.66% |

Specifically, the wrinkled morphologies resulted in a drastic reduction in CV values when detecting L. pneumophila cultures, decreasing from 65.50% to 18.22% at higher concentrations and from 412.61% to 39.23% at lower concentrations. This 2-log improvement in detection sensitivity and more consistent measurement are attributed to the higher density of RFDs within the wrinkled matrices, paired with a larger surface area, which offers greater access to embedded RFDs. Finally, the wrinkled phage microdot arrays were applied for a blind test of real-world cooling tower water samples (coded as sample 1, 2, 3) acquired from industrial plants in Canada (FIG. 28i). Sample 1 and 2 showed significant fluorescent decrease of 62.2% and 32.7%, respectively, while the reduction of sample 3 was near 0%, indicating that the cooling tower sample 1-2 posed a risk for L. pneumophila contaminations. This output matched with the culture results, where L. pneumophila was positively identified in samples 1 and 2, while sample 3 was culture negative. The combined results prove the effectiveness and accuracy of phage-RFD microsensors for the detection of L. pneumophila in real world samples.

### 3. Conclusion

Two general approaches exist for creating artificial tiered structures, namely top-down, or reductive manufacturing, and bottom-up, or additive manufacturing. No method in either category, however, has succeeded in creating hierarchical structures with biomolecules as the building blocks, and thus biological entities have remained the exclusive engineers of such constructs. In addition, and as a direct consequence of this gap in technology, artificial wrinkled structures made with a single material have, to date, all been 1- or 2-level structures^{43,44}, and 3-level or higher hierarchies have mainly been achieved by incorporating engineered nanoparticles or additional polymer/inorganic coatings in the wrinkled structures^{12,16,45,46} Artificial 4-level hierarchies made with a single material have been challenging to achieve. The HPCD method addresses this clear technology gap and, in doing so, unleashes the power of biological entities as building blocks for bioinspired hierarchical structures. This method not only makes it possible to use natural biological entities as building blocks for wrinkled material, but it also enables the creation of higher levels of hierarchy through the preservation of the sophisticated nanoarchitecture of the biological building blocks. Employing nanofilamentous viral nanoparticles as building blocks imparts an additional level of hierarchy to the final wrinkled structure because of their tendency for nanocrystalline self-organization; an ordered structure that other methods of wrinkling fail to preserve. This technology, therefore, allows us to draw from the remarkable diversity of the biological world for creating sophisticated bioinspired architectures in biomaterials and interfaces. Viral nanoparticles offer immense diversity in virion nanoarchitecture, geometrical shape, size, and surface chemistry, in addition to the ability to self-propagate into pseudo monodisperse batches and the potential for self-organization. All of these properties go well above and beyond what engineered nanoparticles, pure or in combination with one another, can ever offer. We demonstrated the power of HPCD method for building directly with natural biological nanoparticles by creating a 4-level hierarchy using a bioink made of phages and a crosslinker (a phage-exclusive network). It should be noted that the few other bioinks reported to date that contain phage, used phages as added bioagents and not as building blocks^{47,48} This phage-exclusive network exhibited highly ordered liquid crystalline alignment, which was immaculately preserved through the wrinkling process. We created two additional layers of hierarchy to what has been achievable in a pure material as a direct consequence of addressing the gap in technology, which could contribute to further advances in nanotechnology for years to come.

Biological building blocks can offer a more powerful functionality, beyond nanoarchitecture and physical intelligence (i.e., self-assembly), namely biorecognition. Biorecognition can be added using biological entities such as antibodies, enzymes, or nucleic acids, all of which are deactivated in high heat used for thermal substrate shrinkage or solvent vapours such as toluene used for solvent based substrate shrinkage. Researchers have thus not been able to incorporate the biorecognition elements directly into the substrate prior to shrinkage; these are usually added after wrinkling, which can result in low and/or inhomogeneous coverage, partially negating the added benefit of wrinkling for increasing the sensing surface area. We demonstrated that our biorecognition element of choice (DNAzyme) remained active during the HPCD process and thus proved that using HPCD shrinkage, functional wrinkles can be created with DNAzyme incorporated directly in the phage matrix during crosslinking. This results in a higher degree of surface coverage and greater homology in three dimensions. It is also a more streamlined process for creating functional wrinkles (one pot vs step-wise). The wrinkle patterns were further verified to be tunable; this is very powerful because the same wrinkle pattern/density may not be optimal for different biorecognition elements (55 nm T7 phage vs. 20-60° A aptamer). Following the same logic, the same wrinkle density may not be suitable for detecting circulating tumor cells in blood (˜10 μm) and small molecule chemical pollutants in water (a few angstroms). Tunability means that, in theory, we can design the shape and wrinkle density that would increase bioavailability of the biological entity or biorecognition molecule of interest, and/or decrease the mass transfer limitations for the analyte being detected based on its size and shape. The strength of HPCD method for creating functional wrinkles lies in the fact that regardless of the size biorecognition element or the pattern/density of the wrinkles, the biorecognition element is homogeneously distributed on the surface and inside the phage matrix. The latter could enable us to make functional wrinkles with controlled release or triggered release behaviour, releasing the biological or therapeutic of choice from the bulk of the wrinkles slowly with time or in response to environmental triggers.

We verified the mechanism of action for HPCD shrinkage to be the decrease in glass transition temperature of the substrate mediated by high pressure carbon dioxide gas. This method can create diverse wrinkled morphologies in 2D or 3D materials where the materials can be in nano, micro, or macro scale, using a wide range of pre-strained polystyrene substrates (which can in theory be of any shape and curvature, flat or 3D). We confirmed that this mechanism works for other polymeric substrates; however, our workflow was constrained. We were able to shrink pre-strained substrates if the substrate's T_{g} could be reduced to below 35° C. by CO₂ gas at 74 bars. We acknowledge that our setup limited the substrates we could verify to those with a specific phase diagram. With a pressurized chamber that could independently control temperature and pressure, it would be possible to investigate a wider range of polymeric substrates. It is also possible that other small molecule gases may have a similar effect on polymeric substrates, as there are a few reports on hydrogen gas acting as a plasticizer⁴⁹, but this needs to be further explored. It is noteworthy that one of the limitations we came across for creating the 4-level structure was the mechanical properties of the substrate. For example, even though PVC could be readily shrunk with HPCD, the material is too soft and can bend during shrinkage, distorting a portion of the phage microdots.

In summary, we addressed a gap in bio/nanotechnology by developing a biologics friendly method for creating bioinspired multi-level tiered structures. This allowed us to preserve the biorecognition ability of DNAzyme and create homogenously functional wrinkled structures in one pot. It also enabled the creation of hierarchy of length scales, which is prevalent in biological constructs, in engineered material/interfaces by adding the physical intelligence of our biological building blocks (phage nanoparticles) as well as their nanoarchitecture to the final construct, thus creating a material with four levels of basic patterns coexisting at different length scales. Other fibrous materials, regardless of nature, biomaterials like microbial nanowires⁵⁰⁻⁵² or semiconductor nanorods⁵³, could potentially acquire similar hierarchical patterns with enhanced biofunctions with this method.

### 4. Experimental Methods

### Phage Propagation, Purification and Concentration

To propagate M13 phage at a large scale, a single colony of Escherichia coli ER2738 (New England Biolabs Ltd., E4104S) was firstly inoculated in 4 mL of sterile LB medium and incubated for 10 hrs at 37° C. with 210 rpm shaking. Subsequently, 2.5 mL of the E. coli culture (˜10⁹ CFU mL⁻¹) and 10 μL of M13 phage (10¹² PFU mL⁻¹, the American Type Culture Collection, 15669-B1) were added to 250 mL of sterile LB broth and incubated overnight at 210 rpm at 37° C. The propagated phage culture was then centrifuged at 7000×g for 15 minutes. After discarding the bacterial pellet, the phage-containing supernatant was then purified by following the procedure described by Sambrook⁵⁴. Briefly, the phage supernatant was mixed in a sterile polyethylene glycol (PEG) solution (20% PEG, 2.5 M NaCl) and stored in a fridge at 4° C. overnight. The following day, the PEG-phage solution was centrifuged at 5000×g and 4° C. for 25 mins to precipitate M13 phages. The pelleted phages were resuspended in 5 mL of sterilized water and gently shaken overnight on a roller at 4° C. The resuspended phage solution was again centrifuged at 5000×g for 30 mins to remove any bacterial remnants. The resulting phage solution was concentrated using 100 kDa and 30 kDa centrifugal filters (Millipore Sigma, Ultra-15) to remove excess water. The final phage concentration was evaluated using the plaque assay method⁵⁵.

### Inkjet Printing of Phage Microgel Array

M13 bacteriophage (1×10¹⁴ PFU mL⁻¹) was mixed with EDC (0.1 M) to constitute the bioink. Optionally, the polystyrene substrates (Grafix shrink film, transparent, purchased from Amazon) were treated with CO₂ plasma for 3 mins through a plasma system (Plasma Etch, Inc.). A Scienion printer was subsequently used to print the bioink onto the substrates under 75% relative humidity, and the ink volume on spot was tuned from 3.25 nL to 13 nL. Then the samples were transferred to a sealed humid box for 12 hrs for phage gelation. Afterwards, the samples were washed by MilliQ water 3 times to remove redundant EDC.

### High Pressure Carbon Dioxide (HPCD) Method

The prestrained polystyrene substrates (with and without phage microarrays) were placed in a Leica critical point dryer (EM CPD300). The process was similar to critical point dry but without the involvement of ethanol. The sample chamber was firstly cooled to 14° C. to allow for the filling with liquid CO₂, and the chamber pressure climbed to the 55-bar range during this phase. Subsequently, the chamber was heated to 35° C. slowly (taking approximately 1 hr). As the temperature rose, the pressure slowly increased to a maximum of 79 bar. As CO₂ transitions to its gaseous phase, the gas was slowly released for 2 hrs until the chamber was back down to 1 bar. Then the substrates were taken out for characterizations and further experiments.

### Scanning Electron Microscopy

Prior to imaging, the samples were dehydrated by air dry. Two types of Scanning Electron Microscopy (SEM) were used to image the microdots. TESCAN VEGA-II LSU SEM was used to image the polystyrene substrate and wrinkled microstructures of the samples, where 10-nm layers of gold were coated onto the samples in advance. A field emission scanning electron microscope (FEI Magellan 400) was used to image the submicron-scale bundles on the wrinkles, where 3-nm layers of Pt were coated onto the samples in advance. The diameter and distance of phage microdots were analyzed by ImageJ based on the SEM images.

### Quantification of Width of Wrinkles

To determine the width of wrinkles, SEM images of wrinkled structures were firstly adjusted for contrast as shown in FIG. 7. At least 50 wrinkles from 5 different images per group were manually selected and measured on ImageJ.

### Atomic Force Microscopy

Atomic force microscopy images were collected using an MFP3D AFM (Asylum Research, Santa Barbara, CA) and a probe tip (Hi'Res-C14, μmash) with a spike radius below 1 nm, a typical spring constant of 5.0 N m⁻¹ and resonance frequency ranging from 110-220 KHz. Igor Pro 6.0 (WaveMetrics, Inc. Lake Oswego, OR) and the Asylum software package (Asylum Research, Santa Barbara, CA) were applied to process AFM images and analyzed surface roughness.

### Inverted Fluorescence Microscopy

Bright field and fluorescent micrographs of the microarrays were obtained using an inverted microscope (Nikon Eclipse Ti2 inverted microscope). Fluorescent micrographs were captured using different optical filter sets according to the applied fluorophore (green channel: ex/em=465/515 nm; orange channel: ex/em=528/590 nm; red channel: ex/em=625/670 nm). The intensity of the light source and the exposure time were consistent. The associated NIS-Elements Analysis software was applied to quantify the fluorescence of microdots.

### Phage-Antibody Microdot Testing

Human/primate IL-6 biotinylated antibody (R&D systems, Ontario, Canada) was mixed with the phage bioink at the concentration of 150 μg mL⁻¹, as the phage-antibody bioink. Following the same bioink printing process and HPCD method, flat and wrinkled phage-antibody microarrays were fabricated. ELISA wash buffer (R&D systems, Ontario, Canada) was used to wash the samples once before incubation with Streptavidin eFluor™ 660 Conjugate (Fisher Scientific, ON, Canada) at 1:1000 v: v in PBS. After 1 h incubation (37° C., 210 rpm), the samples were washed twice with the wash buffer and characterized by inverted fluorescence microscopy.

L. pneumophila Propagation and Quantification

L. pneumophila strain Togus-1 (serotype 2, ATCC 33154) was cultured onto phosphate buffered charcoal yeast extract (BYCE) agar plates from a frozen stock. The plates were incubated at 37° C. for 3 days. Single colonies were then inoculated within 5 mL of buffered yeast extract and grown to an OD₆₀₀ of 1.1. Quantification was performed via serial dilution plating of the resultant culture on BYCE plates at 37° C. for 3 days, wherein a concentration of ˜10⁹ CFU mL⁻¹ was subsequently observed. Crude extracellular mixtures were prepared from these cultures via centrifugation at 6000×g at 4° C. for 5 min. The resultant supernatant was collected, processed through a 0.22 μm filter, and stored at −80° C. until use.

### Phage-RFD Microdot Testing

L. pneumophila-specific RFD was synthesized as reported by Rothenbroker et al⁴⁰ (sequence: 5′-/Cy5/CTATGAACTGACTrATGACCTCACTACCAAGCAAGCATGGACAATACCGAGC CTTTCATTTCAGCCGATCATACCTCAATGTAGATAAGCACATCTTGTCATCGGAGG CTTAG/AmMO/-3′; SEQ ID NO: 1), and incorporated into the microdot solution prior to nanoplotter-mediated deposition to induce homogenous distribution within the three-dimensional structure. Planar, two-dimensional sensors involved EDC/NHS-mediated attachment to CO₂ plasma-activated glass substrates. Fluorescence characterization involved the use of a Nikon AIR Upright Confocal microscope. All sensing samples were imaged prior to testing to establish baseline fluorescence. Positive control samples were incubated with 1 M NaOH, while negative control samples were incubated with a reaction buffer composed of 30 mM MgCl₂—an agent necessary for RFD function. L. pneumophila testing employed serially diluted crude extracellular mixture samples diluted in 30 mM MgCl₂ reaction buffer. Following incubation for 24 hrs at 37° C., samples were briefly washed in deionized water, air dried, and imaged. Post-testing fluorescence intensity was compared to the baseline, pre-testing intensity of a given sample to obtain percent decrease. Target specific signal values represent the fluorescence decrease of a given test sample less the non-specific fluorescence decrease of a paired control sample.

### REFERENCES

1. Koçer, G. et al. Light-Responsive Hierarchically Structured Liquid Crystal Polymer Networks for Harnessing Cell Adhesion and Migration. Adv. Mater. 29, (2017).
2. Moutos, F. T., Freed, L. E. & Guilak, F. A biomimetic three-dimensional woven composite scaffold for functional tissue engineering of cartilage. Nat. Mater. 6, 162-167 (2007).
3. Wang, X. et al. Microfluidic 3D Printing Responsive Scaffolds with Biomimetic Enrichment Channels for Bone Regeneration. Adv. Funct. Mater. 31, (2021).
4. Cutler, S. & Wheeldon, I. Plant hormone sensors as scaffolds for biosensor design. Nat. Biotechnol. 40, 1772-1773 (2022).
5. Bian, F. et al. Bioinspired Perovskite Nanocrystals-Integrated Photonic Crystal Microsphere Arrays for Information Security. Adv. Sci. 9, 1-9 (2022).
6. Zhu, C., Tian, L., Liao, J., Zhang, X. & Gu, Z. Fabrication of Bioinspired Hierarchical Functional Structures by Using Honeycomb Films as Templates. Adv. Funct. Mater. 28, 1-8 (2018).
7. Tian, L., He, L., Jackson, K., Mahabir, R. & Hosseinidoust, Z. Bacteria repellent protein hydrogel decorated with tunable, isotropic, nano-on-micro hierarchical microbump array. Chem. Commun. 57, 10883-10886 (2021).
8. Wei, Y., Chen, S., Yuan, X., Wang, P. & Liu, L. Multiscale Wrinkled Microstructures for Piezoresistive Fibers. Adv. Funct. Mater. 26, 5078-5085 (2016).
9. Dong, G. et al. Periodic Wrinkle-Patterned Single-Crystalline Ferroelectric Oxide Membranes with Enhanced Piezoelectricity. Adv. Mater. 32, 2004477 (2020).
10. Kang, H. et al. Fingerprint-Inspired Conducting Hierarchical Wrinkles for Energy-Harvesting E-Skin. Adv. Funct. Mater. 29, 1-10 (2019).
11. Chan, E. P., Smith, E. J., Hayward, R. C. & Crosby, A. J. Surface wrinkles for smart adhesion. Adv. Mater. 20, 711-716 (2008).
12. Imani, S. M. et al. Flexible Hierarchical Wraps Repel Drug-Resistant Gram-Negative and Positive Bacteria. ACS Nano 14, 454-465 (2020).
13. Hallett, M. B., Von Ruhland, C. J. & Dewitt, S. Chemotaxis and the cell surface-area problem. Nat. Rev. Mol. Cell Biol. 9, 662 (2008).
14. Li, B., Cao, Y. P., Feng, X. Q. & Gao, H. Surface wrinkling of mucosa induced by volumetric growth: Theory, simulation and experiment. J. Mech. Phys. Solids 59, 758-774 (2011).
15. Lin, P. C., Vajpayee, S., Jagota, A., Hui, C. Y. & Yang, S. Mechanically tunable dry adhesive from wrinkled elastomers. Soft Matter 4, 1830-1835 (2008).
16. De France, K. J. et al. 2.5D Hierarchical Structuring of Nanocomposite Hydrogel Films Containing Cellulose Nanocrystals. ACS Appl. Mater. Interfaces 11, 6325-6335 (2019).
17. Jung, W. Bin, Cho, K. M., Lee, W. K., Odom, T. W. & Jung, H. T. Universal Method for Creating Hierarchical Wrinkles on Thin-Film Surfaces. ACS Appl. Mater. Interfaces 10, 1347-1355 (2018).
18. Abbaszadeh Amirdehi, M., Saem, S., Zarabadi, M. P., Moran-Mirabal, J. M. & Greener, J. Microstructured Anodes by Surface Wrinkling for Studies of Direct Electron Transfer Biofilms in Microbial Fuel Cells. Adv. Mater. Interfaces 5, 1-8 (2018).
19. Chan, E. P. & Crosby, A. J. Fabricating microlens arrays by surface wrinkling. Adv. Mater. 18, 3238-3242 (2006).
20. Kim, H. S. & Crosby, A. J. Solvent-responsive surface via wrinkling instability. Adv. Mater. 23, 4188-4192 (2011).
21. Tanford, C. Protein denaturation. Adv. Protein Chem. 23, 121-282 (1968).
22. Tian, L. et al. Bacteriophage-Built Gels as Platforms for Biomedical Applications. Can. J. Chem. Eng. 100, 2191-2203 (2022).
23. Tian, L. et al. Phage display for the detection, analysis, disinfection, and prevention of Staphylococcus aureus. Smart Med. 1, e20220015 (2022).
24. Chung, W. J. et al. Biomimetic self-templating supramolecular structures. Nature 478, 364-368 (2011).
25. Jackson, K., Peivandi, A., Fogal, M., Tian, L. & Hosseinidoust, Z. Filamentous Phages as Building Blocks for Bioactive Hydrogels. ACS Appl. Bio Mater. 4, 2262-2273 (2021).
26. Tian, L. et al. High-throughput fabrication of antimicrobial phage microgels and example applications in food decontamination. Nat. Protoc. 19, 1591-1622 (2024).
27. Oh, J. W. et al. Biomimetic virus-based colourimetric sensors. Nat. Commun. 5, 1-8 (2014).
28. Jung, S. M., Qi, J., Oh, D., Belcher, A. & Kong, J. M13 Virus Aerogels as a Scaffold for Functional Inorganic Materials. Adv. Funct. Mater. 27, 1603203 (2017).
29. Peivandi, A., Tian, L., Mahabir, R. & Hosseinidoust, Z. Hierarchically Structured, Self-Healing, Fluorescent, Bioactive Hydrogels with Self-Organizing Bundles of Phage Nanofilaments. Chem. Mater. 31, 5442-5449 (2019).
30. Peivandi, A. et al. Inducing Microscale Structural Order in Phage Nanofilament Hydrogels with Globular Proteins. ACS Biomater. Sci. Eng. 8, 340-347 (2022).
31. Tian, L. et al. Self-assembling nanofibrous bacteriophage microgels as sprayable antimicrobials targeting multidrug-resistant bacteria. Nat. Commun. 13, 7158 (2022).
32. Souza, G. R. et al. Three-dimensional tissue culture based on magnetic cell levitation. Nat. Nanotechnol. 5, 291-296 (2010).
33. Wang, W. C. V., Kramer, E. J. & Sachse, W. H. Effects of High-Pressure CO2 on the Glass Transition Temperature and Mechanical Properties of Polystyrene. J. Polym. Sci. Part A-2, Polym. Phys. 20, 1371-1384 (1982).
34. Kusmanto, F., Billham, M. & Hornsby, P. Polymer plasticization using supercritical carbon dioxide: Experiment and Modeling. J. Vinyl Addit. Technol. 14, 163-166 (2008).
35. Chow, T. S. Molecular Interpretation of the Glass Transition Temperature of Polymer-Diluent Systems. Macromolecules 13, 362-364 (1980).
36. Gutiérrez, C., Rodríguez, J. F., Gracia, I., De Lucas, A. & Teresa García, M. Modification of polystyrene properties by CO2: Experimental study and correlation. J. Appl. Polym. Sci. 132, 1-9 (2015).
37. Ryu, H. J., Hwang, J., Kim, J. & Lee, J.-H. Dependence of the birefringence of polystyrene film on the stretching conditions. Appl. Opt. 57, 268 (2018).
38. World Health Organizatio. Legionellosis. (2022). Available at: https://www.who.int/news-room/fact-sheets/detail/legionellosis. (Accessed: 6 Jan. 2023)
39. Khan, S. et al. DNAzyme-Based Biosensors: Immobilization Strategies, Applications, and Future Prospective. ACS Nano 15, 13943-13969 (2021).
40. Rothenbroker, M. et al. Selection and Characterization of an RNA-Cleaving DNAzyme Activated by Legionella pneumophila. Angew. Chemie-Int. Ed. 60, 4782-4788 (2021).
41. Qian, S. et al. Detecting Legionella pneumophila in Cooling Tower Water Samples with a DNAzyme/Bead-Based Fluorescence Assay. Anal. Sens. e202300020 (2023). doi: 10.1002/anse.202300020
42. Prasad, A. et al. Advancing in Situ Food Monitoring Through A Smart Lab-in-a-Package System Demonstrated by The Detection of Salmonella in Whole Chicken. Adv. Mater. (2023). doi: 10.1002/adma.202302641
43. Li, M. et al. Microarchitecture for a Three-Dimensional Wrinkled Surface Platform. Adv. Mater. 27, 1880-1886 (2015).
44. Hou, H., Yin, J. & Jiang, X. Reversible Diels-Alder Reaction To Control Wrinkle Patterns: From Dynamic Chemistry to Dynamic Patterns. Adv. Mater. 28, 9126-9132 (2016).
45. Efimenko, K. et al. Nested self-similar wrinkling patterns in skins. Nat. Mater. 4, 293-297 (2005).
46. Moon, M. W. et al. Wrinkled hard skins on polymers created by focused ion beam. Proc. Natl. Acad. Sci. U.S.A 104, 1130-1133 (2007).
47. Lee, D.-Y. et al. Phage as versatile nanoink for printing 3-D cell-laden scaffolds. Acta Biomater. 29, 112-124 (2016).
48. Anany, H. et al. Print to detect: a rapid and ultrasensitive phage-based dipstick assay for foodborne pathogens. Anal. Bioanal. Chem. 410, 1217-1230 (2018).
49. RAHMAN, M. & BRAZEL, C. The plasticizer market: an assessment of traditional plasticizers and research trends to meet new challenges. Prog. Polym. Sci. 29, 1223-1248 (2004).
50. Malvankar, N. S. et al. Tunable metallic-like conductivity in microbial nanowire networks. Nat. Nanotechnol. 6, 573-579 (2011).
51. Lovley, D. R. Microbial nanowires. Curr. Biol. 32, R110-R112 (2022).
52. Lekbach, Y. et al. Microbial nanowires with genetically modified peptide ligands to sustainably fabricate electronic sensing devices. Biosens. Bioelectron. 226, 115147 (2023).
53. Greener, J., Van Der Loop, T. H., Paquet, C., Scholes, G. & Kumacheva, E. A study of simultaneous patterning and alignment of semiconductor nanorods via polymerization-induced phase separation. Langmuir 25, 3173-3177 (2009).
54. McCuen, R. Molecular cloning: a laboratory manual. Cold spring Harb. Lab. Press 186, 182-183 (1990).
55. Kropinski, A. M., Mazzocco, A., Waddell, T. E., Lingohr, E. & Johnson, R. P. Enumeration of bacteriophages by double agar overlay plaque assay. Bacteriophages: Methods and Protocols, Volume 1: Isolation, Characterization, and Interactions 501, (2009).

### FOOTNOTE REFERENCES

1. Grabarek, Z. & Gergely, J. Zero-length crosslinking procedure with the use of active esters. Anal. Biochem. 185, 131-135 (1990).
2. Courchesne, N. M. D. et al. Assembly of a bacteriophage-based template for the organization of materials into nanoporous networks. Adv. Mater. 26, 3398-3404 (2014).
3. Toratani, H. & Takamizawa, K. Effect of pressure on the relation between glass transition temperature and molecular weight for monodispersed polystyrenes. Polym. J. 26, 797-803 (1994).
4. Wang, W. C. V., Kramer, E. J. & Sachse, W. H. Effects of High-Pressure CO2 on the Glass Transition Temperature and Mechanical Properties of Polystyrene. J. Polym. Sci. Part A-2, Polym. Phys. 20, 1371-1384 (1982).
5. Kusmanto, F., Billham, M. & Hornsby, P. Polymer plasticization using supercritical carbon dioxide: Experiment and Modeling. J. Vinyl Addit. Technol. 14, 163-166 (2008).
6. Chow, T. S. Molecular Interpretation of the Glass Transition Temperature of Polymer-Diluent Systems. Macromolecules 13, 362-364 (1980).
7. Gutiérrez, C., Rodríguez, J. F., Gracia, I., De Lucas, A. & Teresa García, M. Modification of polystyrene properties by CO₂: Experimental study and correlation. J. Appl. Polym. Sci. 132, 1-9 (2015).
8. Jacobs, L. J. M., Kemmere, M. F. & Keurentjes, J. T. F. Sustainable polymer foaming using high pressure carbon dioxide: A review on fundamentals, processes and applications. Green Chem. 10, 731-73 (2008).
9. Berens, A. R., Huvard, G. S., Korsmeyer, R. W. & Kunig, F. W. Application of compressed carbon dioxide in the incorporation of additives into polymers. J. Appl. Polym. Sci. 46, 231-242 (1992).
10. Fleming, O. S. & Kazarian, S. G. Polymer Processing with Supercritical Fluids. Supercrit. Carbon Dioxide Polym. React. Eng. 42, 205-238 (2006).
11. Choy, E. H. et al. Translating IL-6 biology into effective treatments. Nat. Rev. Rheumatol. 16, 335-345 (2020).
12. Ridker, P. M. et al. IL-6 inhibition with ziltivekimab in patients at high atherosclerotic risk (RESCUE): a double-blind, randomised, placebo-controlled, phase 2 trial. Lancet 397, 2060-2069 (2021).
13. Jones, S. A., Scheller, J. & Rose-John, S. Therapeutic strategies for the clinical blockade of IL-6/gp130 signaling. J. Clin. Invest. 121, 3375-3383 (2011).

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

1. A biomolecule-based wrinkled network.

2. The wrinkled network of claim 1, wherein the wrinkled network is nano-reticular.

3. The wrinkled network of claim 1, comprising a hierarchical architecture.

4. The wrinkled network of claim 3, comprising four levels of hierarchy.

5. The wrinkled network of claim 1, wherein the wrinkled network comprises a tunable pattern of wrinkle shape and/or density.

6. The wrinkled network of claim 1, wherein the biomolecule comprises a protein, a nucleic acid, a bacteria, a virus, a phage, or any combination thereof.

7. The wrinkled network of claim 1, further comprising a cargo.

8. The wrinkled network of claim 7, wherein the cargo is homogenously distributed throughout the wrinkled network.

9. The wrinkled network of claim 7, wherein the cargo comprises a biorecognition element.

10. The wrinkled network of claim 9, wherein the biorecognition element comprises an antibody, an enzyme, an aptamer, a cell, a nucleic acid, a protein, or any combination thereof.

11. The wrinkled network of claim 10, wherein the enzyme comprises a deoxyribozyme.

12. The wrinkled network of claim 9, wherein the biorecognition element detects an enzyme, antibody, antigen, nucleic acid, cell, aptamer, tissue, microorganism, organelle, cell receptor, or any combination thereof.

13. The wrinkled network of claim 1 configured in an antimicrobial material, a microarray, a wearable patch, a smart textile, a wearable device, a flexible electronic, an adhesive/repellent surface, a biosensor, a bioassays, a cell culture substrate, a biomimetic material, a surface-enhanced Raman spectroscopy, an electrode, a catalyst, a drug delivery device, or an energy storage material.

14. A method for wrinkling a biomolecule-based network disposed on a substrate, the method comprising shrinking the substrate without using heat or solvent.

15. The method of claim 14, wherein shrinking the substrate comprises applying high pressure plasticizer gas to the substrate.

16. The method of claim 15, wherein the high pressure plasticizer gas comprises carbon dioxide and/or hydrogen.

17. The method of claim 14, wherein the biomolecule comprises a protein, a nucleic acid, a bacteria, a virus, a phage, or any combination thereof.

18. The method of claim 14, further comprising printing the biomolecule-based network on the substrate.

19. The method of claim 14, further comprising crosslinking the biomolecule-based network prior to shrinking the substrate.

20. The method of claim 14, further comprising drying the biomolecule-based network prior to shrinking the substrate.

21. The method of claim 14, wherein the wrinkle shape, density, and/or homogeneity are tunable by adjusting the hydrophobicity of the substrate and/or by adjusting the biomolecule concentration in the network.

22. The method of claim 21, wherein the substrate comprises a prestrained polymeric material.

23. The method of claim 22, wherein the prestrained polymeric material comprises polystyrene and/or polyvinyl chloride.

24. The method of claim 14, wherein the method is a one pot process.

25. An antimicrobial material, a microarray, a wearable patch, a smart textile, a wearable device, a flexible electronic, an adhesive/repellent surface, a biosensor, a bioassays, a cell culture substrate, a biomimetic material, a surface-enhanced Raman spectroscopy, an electrode, a catalyst, a drug delivery device, or an energy storage material comprising the wrinkled network of claim 1.
